(19) United States
(12) Patent Application Publication
(10) Pub. No.: US 2010/0183543 A1
(43) Pub. Date: Jul. 22, 2010

(54) METHOD FOR THE TREATMENT OF RADIATION-INDUCED NEUTROPENIA BY ADMINISTRATION OF A MULTI-PEGYLATED GRANULOCYTE COLONY STIMULATING FACTOR (G-CSF) VARIANT

(75) Inventors: Grant Yonehiro, Woodside, CA (US); Thomas J. MacVittie, Silver Spring, MD (US)

Correspondence Address: MAXYGEN, INC.;INTELLECTUAL PROPERTY DEPARTMENT, 515 GALVESTON DRIVE, REDWOOD CITY, CA, 94063, US

(73) Assignee: MAXYGEN, INC.

(21) Appl. No.: 12/563,022

(22) Filed: Sep. 18, 2009

Related U.S. Application Data
(60) Provisional application No. 61/098,569, filed on Sep. 19, 2008.

(51) Int. Cl. A61K 38/19; A61K 38/20; A61P 37/04

(52) U.S. Cl. 424/85.2; 424/85.1

(57) ABSTRACT

The invention relates to a method for treating or preventing radiation-induced neutropenia in a patient exposed to radiation by administering to the patient a multi-PEGylated granulocyte colony stimulating factor (G-CSF) variant.

### CROSS-REFERENCE TO RELATED APPLICATIONS

Pursuant to 35 U.S.C. §119(e), this application claims the benefit of U.S. Provisional Application Ser. No. 61/098,569 filed on Sep. 19, 2008, the disclosure of which is incorporated by reference herein in its entirety for all purposes.

### FIELD OF THE INVENTION

The present invention relates to a method for treating or preventing radiation-induced neutropenia by administering a multi-PEGylated granulocyte colony stimulating factor (G-CSF) variant.

### BACKGROUND OF THE INVENTION

The process by which white blood cells grow, divide and differentiate in the bone marrow is called hematopoiesis (Dexter and Spooncer, Ann. Rev. Cell. Biol., 3:423, 1987). Each of the blood cell types arises from pluripotent stem cells. There are generally three classes of blood cells produced in vivo: red blood cells (erythrocytes), platelets and white blood cells (leukocytes), the majority of the latter being involved in host immune defense. Proliferation and differentiation of hematopoietic precursor cells are regulated by a family of cytokines, including colony-stimulating factors (CSFs) such as G-CSF and interleukins (Arai et al., Ann. Rev. Biochem., 59:783-836, 1990). The principal biological effect of G-CSF in vivo is to stimulate the growth and development of certain white blood cells known as neutrophilic granulocytes or neutrophils (Welte et al., PNAS 82:1526-1530, 1985; Souza et al., Science, 232:61-65, 1986). When released into the blood stream, neutrophilic granulocytes function to fight bacterial and other infections.

The amino acid sequence of human G-CSF (hG-CSF) was reported by Nagata et al. (Nature 319:415-418, 1986). hG-CSF is a monomeric protein that dimerizes the G-CSF receptor by formation of a 2:2 complex of 2 G-CSF molecules and 2 receptors (Horan et al., Biochemistry 35(15): 4886-96, 1996). In a more recent publication (PNAS 103:3135-3140, 2006), Tamada et al. described a crystal structure of the signaling complex between human G-CSF and a ligand binding region of the GCSF receptor.

Leukopenia (a reduced level of white blood cells) and neutropenia (a reduced level of neutrophils) are disorders that result in an increased susceptibility to various types of infections. For patients with severe neutropenia (also termed febrile neutropenia), exhibited by an absolute neutrophil count (ANC) below about 500 cells/mm³, even relatively minor infections can be serious and even life-threatening. Recombinant human G-CSF (rhG-CSF) is often used for treating and preventing various forms of leukopenia and neutropenia. Preparations of rhG-CSF are commercially available, e.g. Neupogen® (Filgrastim), which is non-glycosylated and produced in recombinant E. coli cells, and Neulasta® (Pegfilgrastim), which has the same amino acid sequence as Neupogen® but contains a single, N-terminally linked 20 kDa polyethylene glycol (PEG) group. This mono-PEGylated rhG-CSF molecule has been shown to have an increased half-life compared to non-PEGylated G-CSF and thus may be administered less frequently than the non-PEGylated G-CSF products, and reduces the duration of neutropenia to about the same number of days as by administration of non-PEGylated G-CSF.

Acute Radiation Syndrome (ARS), also known as radiation sickness or radiation illness, encompasses a set of complex pathophysiological processes precipitated by exposure to high doses of radiation affecting the hematologic, gastrointestinal and cardiovascular systems. ARS generally occurs after whole-body or significant partial-body irradiation of about 0.7 to 1 gray (Gy) or more delivered over a relatively short time period (Waselenko J. K. et al., Annals of Internal Medicine 140(12):1037-1051, 2004; Jarrett D. G. et al., Radiation Measurements 42:1063-1074, 2007). The latency, severity, and duration of the various manifestations of ARS are a function of the radiation dose, dose rate, and type of radiation, as well as the heterogeneity or homogeneity of the precipitating exposure.

ARS follows a somewhat predictable course and is characterized by symptoms which are manifestations of the specific reaction of various cells, tissues, and organ systems to radiation (see, e.g., Waselenko et al., supra, particularly FIG. 1, Tables 1-3 and Table 5 therein). Symptoms associated with ARS include nausea, vomiting, diarrhea, neutropenia, skin burns and sores, fatigue, dehydration, inflammation, hair loss, ulceration of the oral mucosa and GI system, xerostomia, and bleeding (e.g., from the nose, mouth and rectum). Cells which replicate at a high rate, such as hematopoietic progenitor cells, spermatocytes, and intestinal crypt cells are most immediately vulnerable to acute radiation exposure. The probability of measurable clinical effects increases as the total dose or dose rate increases. However, a total radiation dose that produces an observable effect after a single rapid exposure may be tolerated with little measurable effect if given over a more prolonged period of time.

Circulating hematopoietic cells and hematopoietic progenitor (bone marrow) cells are among the most highly radiosensitive cells. A common underlying cause for the symptoms associated with radiation sickness is the effect of radiation on such cells. The hematopoietic syndrome (H-ARS) is seen in humans exposed to significant partial-body or whole-body radiation levels generally exceeding about 0.7-1 Gy (Jarrett et al., supra; Waselenko et al., supra), and is rarely clinically significant below this level. Mitotically active hematopoietic progenitor cells have a limited capacity to divide after a whole-body radiation dose of 2 to 3 Gy. The hematopoietic syndrome of ARS is characterized by reductions in blood cell numbers—white blood cells (WBC; neutrophils and lymphocytes), platelets (also called thrombocytes) and red blood cells (RBC)—with potentially clinically significant outcomes. Exposure to ionizing radiation may lead to decreases in WBC count, which manifests as neutropenia (reduction in neutrophils/granulocytes) and lymphopenia (reduction in lymphocytes). RBC decreases may result in anemia, whereas platelet reduction may lead to thrombocytopenia. The kinetics of radiation-induced neutropenia, thrombocytopenia and anemia depend on the dose received, the dose rate, and the extent to which the body is irradiated (Waselenko et al., supra). Radiation-induced damage to cellular production in the bone marrow begins at the time of exposure. While most bone marrow progenitor cells are susceptible to cell death after sufficiently high radiation doses, sub-populations of stem cells or accessory cells have been found to be more radioresistant, presumably because of their noncycling (G₀) state, which may play an important role in recovery of hematopoiesis after exposure to potentially lethal doses (Waselenko et al., supra).

Radiation effects also depend on the amount of body surface area exposed. It is believed the human body can absorb a single dose of up to about 2 Gy over the whole body area without immediate risk of death. A dose over about 2 Gy, if untreated, leads to probable or certain death due to bone marrow failure. A whole-body dose of about 8 Gy or more given over a short period of time is almost certainly fatal. In contrast, tens of Gy can be tolerated when delivered over a longer period of time, and/or to a small volume of tissue (as in, e.g., for cancer therapy).

Radiation-induced neutropenia increases the susceptibility to life threatening infection by saprophytic and pathogenic organisms, and diminishes immune resistance to bacterial spread in subcutaneous tissues and from breaks in the integrity of the intestinal wall. This susceptibility to infection and sepsis is the primary cause of mortality in subjects with exposures to ionizing radiation in the 2-8 Gy range. Concurrent with neutropenia, varying degrees of thrombocytopenia may also be observed. Severe thrombocytopenia may increase susceptibility to life-threatening bleeding if left untreated.

Radiation-induced neutropenia associated with ARS leads to significant mortality and morbidity in patients exposed to high levels of radiation via, for example, a nuclear incident or accidental radiation exposure. There is a need for long-acting G-CSF products, in particular multi-PEGylated G-CSF, which may safely be administered to reduce radiation-induced neutropenia associated with ARS, and for methods for treatment and prevention of radiation-induced neutropenia using such G-CSF products.

### BRIEF DESCRIPTION OF THE INVENTION

The object of the present invention is to provide a method of treating or preventing neutropenia in patients exposed to radiation, e.g., as a consequence of a nuclear explosion or accidental radiation exposure, to enhance survivability by decreasing the duration and/or severity of radiation-induced neutropenia and thus decreasing the risk of life-threatening infection in such patients.

One aspect of the invention thus relates to a method for treating or preventing neutropenia in a patient subjected to radiation exposure, comprising administering to said patient a multi-PEGylated G-CSF variant in an amount effective to reduce radiation-induced neutropenia, such as radiation-induced neutropenia associated with the acute radiation syndrome (ARS), e.g., the hematopoietic syndrome of ARS (H-ARS).

A further aspect of the invention relates to a multi-PEGylated G-CSF variant for treating or preventing neutropenia by means of the method described herein. This aspect of the invention thus relates to a multi-PEGylated G-CSF variant for the treatment of radiation-induced neutropenia. This aspect of the invention also relates to a multi-PEGylated G-CSF variant for treating or preventing neutropenia in a patient exposed to radiation by administering the multi-PEGylated G-CSF variant to the patient.

A further aspect of the invention relates to use of a multi-PEGylated G-CSF variant for the preparation of a medicament for treating or preventing radiation-induced neutropenia by means of the method described herein. This aspect of the invention thus relates to use of a multi-PEGylated G-CSF variant for the preparation of a medicament for treating or preventing radiation-induced neutropenia in a patient exposed to radiation, wherein the multi-PEGylated G-CSF variant is administered to the patient in an amount effective to reduce radiation-induced neutropenia. This aspect of the invention also relates to use of a multi PEGylated G-CSF variant for the preparation of a medicament for the treatment of radiation-induced neutropenia. This aspect of the invention also relates to use of a multi-PEGylated G-CSF variant for the preparation of a medicament for treating or preventing radiation-induced neutropenia in a patient receiving exposed to radiation by administering the multi-PEGylated G-CSF variant to the patient.

In some embodiments, the multi-PEGylated G-CSF variant is administered to the patient in an amount effective to reduce the duration of severe neutropenia in a group treated with the multi-PEGylated G-CSF variant, relative to a group not treated with the multi-PEGylated G-CSF variant, in an animal model system (such as, a non-human primate model system) of radiation-induced neutropenia. In other embodiments, the multi-PEGylated G-CSF variant is administered to the patient in an amount effective to increase the number of survivors 30 days or 60 days post-radiation exposure in a group treated with the multi-PEGylated G-CSF variant, relative to a group not treated with the multi-PEGylated G-CSF variant, in an animal model system (such as, a non-human primate model system) of radiation-induced neutropenia.

These and other aspects and features of the invention will become more fully apparent when the following detailed description is read in conjunction with the accompanying figures.

### BRIEF DESCRIPTION OF THE DRAWINGS

FIG. 1 shows a 60-day hematopoietic syndrome lethality dose response relationship in rhesus monkeys, presented as probit percent lethality vs TBI dose in grays (Gy) on a log scale. The resulting LD50/60 value for rhesus macaques exposed to 2 MV LINAC photons and receiving supportive care is indicated as LD50_{LINAC} (with the 95% confidence interval in brackets [ ]). This figure also shows two historical data sets showing the TBI dose response and calculated LD50/30 values (with 95% confidence interval in brackets [ ], of rhesus macaques exposed to Co-60 gamma and 2 MV X-radiation (denoted LD50_{Co60} and LD50_{Xray}, respectively). Animals in the historical studies did not receive supportive care.

FIG. 2 shows the timecourse of the change in mean absolute neutrophil count (ANC) in rhesus monkeys after exposure to total-body irradiation at doses which approximate the LD30/60 (720 centigray (cGy)), LD50/60 (755 cGy), and LD70/60 (805 cGy), and given supportive care.

FIG. 3 demonstrates that an exemplary multi-PEGylated G-CSF variant of the invention (identified herein as “Maxy-G21”) improves neutrophil recovery in non-human primates following radiation exposure relative to mono-PEGylated rhG-CSF. Absolute neutrophil counts (ANC) in rhesus monkeys were determined following 600 cGy (6.00 Gy) irradiation and administration of Maxy-G21, Neulasta®, or control (sera) one day post-irradiation. Severe neutropenia (ANC <500 μL) is indicated by the horizontal line.

FIG. 4 shows the pharmacokinetic (PK) profile of 600 cGy-irradiated rhesus monkeys dosed one day post-irradiation with either 300 μg/kg of Maxy-G21 or 300 μg/kg Neulasta®.

FIG. 5 shows a Kaplan-Meier Survival Curve of mice exposed to 776 cGy radiation and subsequently treated with an exemplary multi-PEGylated G-CSF variant of the invention (“G34”, also identified herein as “Maxy-G34”)) or with diluent (“vehicle”). C57BL/6 mice were irradiated and then injected subcutaneously with G34 (1.0 mg/kg=20 μg/20 gm mouse) at 24 hr and 7 days post-exposure (open diamonds), or 24 hr, 7 days, and 14 days post exposure (open squares). Control mice were injected at 24 hr, 7 days, and 14 days post-exposure (closed triangles) with diluent. The mice were not treated with antibiotics.

FIG. 6 shows a Kaplan-Meier Survival Curve of mice exposed to 796 cGy radiation and subsequently treated with an exemplary multi-PEGylated G-CSF variant of the invention (“G34”, also identified herein as “Maxy-G34”)) or with diluent (“vehicle”). C57BL/6 mice were irradiated and then injected subcutaneously with G34 (1.0 mg/kg=20 μg/20 gm mouse) at 24 hr and 7 days post-exposure (open diamonds), or 24 hr, 7 days, and 14 days post-exposure (open squares). Control mice were injected at 24 hr, 7 days, and 14 days post-exposure (closed triangles) with vehicle. The mice were not treated with antibiotics.

### DEFINITIONS

In the description and claims below, the follow definitions apply.

The terms “polypeptide” or “protein” may be used interchangeably herein to refer to polymers of amino acids, without being limited to an amino acid sequence of any particular length. These terms are intended to include not only full-length proteins but also e.g. fragments or truncated versions, variants, domains, etc. of any given protein or polypeptide.

A “G-CSF polypeptide” is a polypeptide having the sequence of human granulocyte colony stimulating factor (hG-CSF) as shown in SEQ ID NO:1, or a variant of hG-CSF that exhibits G-CSF activity. The “G-CSF activity” may be the ability to bind to a G-CSF receptor (Fukunaga et al., J. Bio. Chem, 265:14008, 1990, which is incorporated herein by reference), but is preferably G-CSF cell proliferation activity, which may, for example, be determined in an in vitro activity assay using the murine cell line NFS-60 (ATCC Number: CRL-1838). A suitable in vitro assay for G-CSF activity using the NFS-60 cell line is described by Hammerling et al. in J. Pharm. Biomed. Anal. 13(1):9-20, 1995, which is incorporated herein by reference. A polypeptide “exhibiting G-CSF activity” is considered to have such activity when it displays a measurable function, for example a measurable cell proliferation activity in an in vitro assay.

A “variant” (e.g., a “G-CSF variant”) is a polypeptide which differs in one or more amino acid residues from a parent polypeptide, where the parent polypeptide is generally one with a native, wild-type amino acid sequence, typically a native mammalian polypeptide, and more typically a native human polypeptide. The variant thus contains one or more substitutions, insertions or deletions compared to the native polypeptide. These may, for example, include truncation of the N- and/or C-terminus by one or more amino acid residues, or addition of one or more amino acid residues at the N- and/or C-terminus, for example, addition of a methionine residue at the N-terminus. The variant will most often differ in up to 15 amino acid residues from the parent polypeptide, such as in up to 12, 10, 8 or 6 amino acid residues. Some G-CSF variants, in particular, have amino acid substitutions in the G-CSF sequence either with or without the addition of a methionine residue at the N-terminus.

The term “modified” G-CSF refers to a G-CSF molecule with either the sequence of human G-CSF or a variant of human G-CSF, which is modified by, e.g., alteration of the glycan structure. For example, the glycan structure of G-CSF may be modified for the purpose of providing glyco-PEGylated G-CSF molecules in which polyethylene glycol moieties are attached to a glycosyl linking group such as a sialic acid moiety as described in WO 2005/055946, which is incorporated herein by reference. Another example of a modified G-CSF molecule is one that contains at least one O-linked glycosylation site that does not exist in the wild-type polypeptide. G-CSF molecules having such non-naturally occurring O-linked glycosylation sites, as well as PEGylation of modified sugars of G-CSF, are described in WO 2005/070138, which is incorporated herein by reference.

Unless otherwise indicated, the term “G-CSF” as used herein is intended to encompass G-CSF molecules with the native human sequence (SEQ ID NO:1) as well as variants of the human G-CSF sequence. In either case, the term “G-CSF” is also intended to include modified G-CSF such as G-CSF glycosylation variants.

A PEGylated G-CSF that “comprises multiple polyethylene glycol moieties” (also referred to herein as a “multi-PEGylated G-CSF”) refers to a G-CSF polypeptide having two or more PEG moieties that are covalently attached either directly or indirectly to an amino acid residue of the polypeptide, in contrast to a “mono-PEGylated G-CSF” which has only one PEG moiety covalently attached to the polypeptide. Suitable attachment sites include, for example, the ε-amino group of a lysine residue or the N-terminal amino group, a free carboxylic acid group (e.g. that of the C-terminal amino acid residue or of an aspartic acid or glutamic acid residue), the thiol group of a cysteine residue, suitably activated carbonyl groups, oxidized carbohydrate moieties and mercapto groups. More information on PEG attachment sites and methods for attachment of PEG moieties to proteins may be found, e.g., in WO 01/51510, WO 03/006501, and the Nektar Advanced PEGylation Catalog 2005-2006 (Nektar Therapeutics), all of which are incorporated herein by reference. Another possibility for PEGylation is to attach PEG moieties to the glycan structures of G-CSF, e.g. by way of glycan modification (see above).

A “multi-PEGylated G-CSF variant” refers to a G-CSF variant having two or more PEG moieties that are covalently attached either directly or indirectly to an amino acid residue of the variant.

In the present application, amino acid names and atom names (e.g. CA, CB, NZ, N, O, C, etc.) are used as defined by the Protein Data Bank (PDB), which is based on the IUPAC nomenclature (IUPAC Nomenclature and Symbolism for Amino Acids and Peptides (residue names, atom names etc.), Eur. J. Biochem., 138, 9-37 (1984) together with their corrections in Eur. J. Biochem., 152, 1 (1985). The term “amino acid residue” is intended to indicate any naturally or non-naturally occurring amino acid residue, in particular an amino acid residue contained in the group consisting of the 20 naturally occurring amino acids, i.e. alanine (Ala or A), cysteine (Cys or C), aspartic acid (Asp or D), glutamic acid (Glu or E), phenylalanine (Phe or F), glycine (Gly or G), histidine (His or H), isoleucine (Ile or I), lysine (Lys or K), leucine (Leu or L), methionine (Met or M), asparagine (Asn or N), proline (Pro or P), glutamine (Gln or Q), arginine (Arg or R), serine (Ser or S), threonine (Thr or T), valine (Val or V), tryptophan (Trp or W), and tyrosine (Tyr or Y) residues.

The terminology used for identifying amino acid positions/substitutions herein is illustrated as follows: F13 indicates position number 13 occupied by a phenylalanine residue in the reference amino acid sequence. F13K indicates that the phenylalanine residue of position 13 has been substituted with a lysine residue. Unless otherwise indicated, the numbering of amino acid residues made herein is made relative to the amino acid sequence of hG-CSF shown in SEQ ID NO:1. Alternative substitutions are indicated with a “/”, e.g. K16R/Q means an amino acid sequence in which lysine in position 16 is substituted with either arginine or glutamine. Multiple substitutions are indicated with a “+”, e.g. K40R+T105K means an amino acid sequence which comprises a substitution of the lysine residue in position 40 with an arginine residue and a substitution of the threonine residue in position 105 with a lysine residue.

The term “functional in vivo half-life” is used in its normal meaning, i.e. the time at which 50% of the biological activity of the test molecule (e.g., PEGylated conjugate) is still present in the body/target organ, or the time at which the activity of the polypeptide or conjugate is 50% of the initial value. “Serum half-life” is defined as the time in which 50% of the conjugate molecules circulate in the plasma or bloodstream prior to being cleared. Alternative terms to serum half-life include “plasma half-life”, “circulating half-life”, “serum clearance”, “plasma clearance” and “clearance half-life”. The test molecule (e.g., PEGylated conjugate) is cleared by the action of one or more of the reticuloendothelial systems (RES), kidney, spleen or liver, by receptor-mediated degradation, or by specific or non-specific proteolysis, in particular by the action of receptor-mediated clearance and renal clearance. Normally, clearance depends on size (relative to the cutoff for glomerular filtration), charge, attached carbohydrate chains, and the presence of cellular receptors for the protein. The functionality to be retained is normally selected from proliferative or receptor-binding activity. The functional in vivo half-life and the serum half-life may be determined by any suitable method known in the art.

The term “increased” as used in reference to in vivo half-life or serum half-life is used to indicate that the half-life of the test molecule, i.e. the multi-PEGylated G-CSF variant, is statistically significantly increased relative to that of a reference molecule, such as a non-conjugated (i.e., non-PEGylated) hG-CSF (e.g. Neupogen®) or preferably, relative to the mono-PEGylated G-CSF Neulasta®, as determined under comparable conditions (typically determined in an experimental animal, such as rat, rabbit, pig or monkey). For instance, the serum half-life (t_{1/2}) of the test molecule may be increased by at least about 1.2× to that of the reference molecule (that is, (t_{1/2} of the test molecule)/(t_{1/2} of the reference molecule)=1.2), e.g. by at least about 1.4×, such as by at least about 1.5 x, e.g. by at least about 1.6×, such as by at least about 1.8×, e.g. by at least about 2.0×, 2.5×, 3×, 5×, or 10× to that of the reference molecule.

The term “AUC” or “Area Under the Curve” is used in its normal meaning, i.e. as the area under the serum concentration versus time curve where the test molecule has been administered to a subject. Once the experimental concentration-time points have been determined, the AUC may conveniently be calculated by a computer program such as GraphPad Prism® (GraphPad Software, Inc.).

The term “increased” as used in reference to the AUC is used to indicate that the AUC of the test molecule, i.e. the multi-PEGylated G-CSF variant, is statistically significantly increased relative to that of a reference molecule, such as a non-conjugated hG-CSF (e.g. Neupogen®) or, preferably, relative to the mono-PEGylated hG-CSF Neulasta®, as determined under comparable conditions (typically determined in an experimental animal, such as rat, rabbit, pig or monkey). For instance, the AUC of the test molecule may be increased by at least about 1.2× to that of the reference molecule (that is, (AUC of the test molecule)/(AUC of the reference molecule)=1.2), e.g. by at least about 1.4×, such as by at least about 1.5×, e.g. by at least about 1.6×, such as by at least about 1.8×, e.g. by at least about 2.0×, 2.5×, 3×, 5×, or 10× to that of the reference molecule.

The term “subject” refers to an animal, such as a mammal, including a non-primate (e.g., a cow, pig, horse, cat, or dog) or a primate (e.g., a monkey, chimpanzee, or human) such as a non-human primate (e.g., a monkey or chimpanzee), or a human. In some instances, the subject is a mammal, such as a human, which has been exposed to radiation. The term “subject” is used interchangeably with “patient” herein.

The term “acute radiation exposure” refers to exposure to radiation which occurs during a short period of time, i.e., under 24 hours (such as, less than 20 hours, less than 16 hours, less than 12 hours, less than 10 hours, less than 8 hours, less than 6 hours, less than 2 hours, less than 1 hour, less than 30 minutes, less than 20 minutes, less than 10 minutes, less than 5 minutes, or less than one minute). Acute radiation exposure may result from a nuclear event (such as, a nuclear explosion); a laboratory or manufacturing accident; exposure during handling of highly radioactive sources over minutes or hours; or accidental or intentional high medicinal doses.

The term “radiation dose” refers to the total amount of radiation absorbed by material or tissues, generally expressed in centigrays (cGy) or grays (Gy).

The term “radiation dose rate” refers to the radiation dose (dosage) absorbed per unit of time.

The term “LDx/y” refers to the average dose of radiation which results in death of x % of subjects by y days. For example, the terms LD50/30 and LD50/60 refer to the average dose of radiation which results in death of 50% of the subjects by 30 or 60 days, respectively.

Various additional terms are defined or otherwise characterized herein.

### DETAILED DESCRIPTION OF THE INVENTION

The present invention provides a method for treating or preventing neutropenia in a patient exposed to radiation, where the method comprises administering to said patient a multi-PEGylated G-CSF variant in an amount effective to reduce radiation-induced neutropenia.

We have found that administration of a multi-PEGylated G-CSF variant is more effective at reducing the duration of radiation-induced neutropenia when compared to administration of a mono-PEGylated hG-CSF (Neulasta®) in an irradiated non-human primate model. The reduction of time to absolute neutrophil recovery (ANC) was also significantly improved as compared to both the control and mono-PEGylated hG-CSF (Neulasta®). As used herein, term “time to ANC recovery” is defined as the number of days starting from day one of chemotherapy until the first of two consecutive days where the subject has counts above 0.5×10⁹ ANC cells/L, i.e., above the defining limit for severe neutropenia. Time to ANC recovery, duration/days of leukopenia, and duration/days of severe neutropenia are all indicative of the period during which a patient exposed to radiation is in an immune suppressed state (the terms “days of neutropenia” and “days of severe neutropenia” are used interchangeably herein). During this period, the patient is vulnerable to infections which may exacerbate other symptoms of acute radiation syndrome and which may lead to mortality. In view of the results described in the examples herein, it is contemplated that administration of the multi-PEGylated G-CSF variant is more effective than administration of a mono-PEGylated hG-CSF (Neulasta®) in reducing the magnitude and duration of radiation-induced neutropenia in a subject.

The method of the invention is effective at reducing the time to ANC recovery, days of leukopenia, and days of neutropenia. At equivalent doses, the method is more effective at reducing the time to ANC recovery, days of leukopenia, and days of neutropenia when compared to mono-PEGylated hG-CSF (Neulasta®).

In accordance with the method of the present invention, the multi-PEGylated G-CSF variant is preferably administered within seven days after radiation exposure. For example, the multi-PEGylated G-CSF variant may administered within about 4 days after radiation exposure, such as within 3 days after radiation exposure, e.g., within 2 days after radiation exposure, such as within 1 day (24 hours) after radiation exposure. Depending on the prognosis of the patient, the multi-PEGylated G-CSF variant may be administered two or more times over the course of a treatment regimen. For example, the multi-PEGylated G-CSF variant may be administered weekly, for e.g. two weeks, three weeks or four weeks. Owing to the superior bioavailability of the multi-PEGylated G-CSF variant compared to non-PEGylated hG-CSF (e.g., Neupogen®) and mono-PEGylated hG-CSF (e.g., Neulasta®), multi-PEGylated G-CSF variant preferably may be administered over longer periods of time, such as, for example, every 10 days, every two weeks, every 18 days, or every three weeks, depending on the prognosis of the patient.

### Multi-PEGylated G-CSF Variant

Multi-PEGylated proteins may be prepared in a number of ways that are well known in the art. The covalent attachment (i.e., conjugation) of polyethylene glycol (PEG) moieties to proteins or polypeptides (“PEGylation”) is a well-known technique for improving the properties of such proteins or polypeptides, in particular pharmaceutical proteins, e.g. in order to improve circulation half-life and/or to shield potential epitopes and thus reduce the potential for an undesired immunogenic response. Numerous technologies based on activated PEG are available to provide attachment of the PEG moiety to one or more groups on the protein. For example, mPEG-succinimidyl propionate (mPEG-SPA, available from Nektar Therapeutics) is generally regarded as being selective for attachment to the N-terminus and ε-amino groups of lysine residues via an amide bond. As noted above, the commercially available PEGylated G-CSF product Neulasta® contains a single 20 kDa PEG moiety attached to the N-terminus of the G-CSF molecule.

In some embodiments, multi-PEGylated G-CSF variants described herein exhibit improved pharmacokinetic parameters, such as an increased serum half-life and/or and an increased area under the curve (AUC), relative to the mono-PEGylated G-CSF Neulasta® (pegfilgrastim) when tested in experimental animals such as rats. In accordance with the present invention, a multi-PEGylated G-CSF variant has been found to be advantageous over the mono-PEGylated G-CSF Neulasta® in an animal model of radiation-induced neutropenia, providing a shorter time-to-recovery and a shorter period of neutropenia/leukopenia at equivalent doses.

In one embodiment, the multi-PEGylated G-CSF variant administered according to the invention may be PEGylated with an amine-specific activated PEG that preferentially attaches to the N-terminal amino group and/or to the ε-amino groups of lysine residues via an amide bond. Examples of amine-specific activated PEG derivatives include mPEG-succinimidyl propionate (mPEG-SPA), mPEG-succinimidyl butanoate (mPEG-SBA) and mPEG-succinimidyl α-methylbutanoate (mPEG-SMB) (available from Nektar Therapeutics; see the Nektar Advanced PEGylation Catalog 2005-2006, “Polyethylene Glycol and Derivatives for Advanced PEGylation”); PEG-SS (Succinimidyl Succinate), PEG-SG (Succinimidyl Glutarate), PEG-NPC (p-nitrophenyl carbonate), and PEG-isocyanate, available from SunBio Corporation; and PEG-SCM, available from NOF Corporation. The polyethylene glycol may be either linear or branched.

Methods for obtaining PEGylated proteins are well known in the art; see e.g. the Nektar Advanced PEGylation Catalog 2005-2006, which is incorporated herein by reference. PEGylated G-CSF variants, and methods for their preparation, are e.g. described in WO 01/51510, WO 03/006501, U.S. Pat. No. 6,646,110, U.S. Pat. No. 6,555,660 and U.S. Pat. No. 6,831,158, each of which are incorporated herein by reference.

In a preferred embodiment, the multi-PEGylated G-CSF variant comprises a PEG moiety attached to the N-terminus and at least one PEG moiety attached to a lysine residue.

In one embodiment, the administered multi-PEGylated G-CSF variant comprises at least one substitution in the hG-CSF sequence of SEQ ID NO:1 to introduce a lysine residue in a position where PEGylation is desired. In particular, the lysine residue may be introduced by way of one or more substitutions selected from the group consisting of T1K, P2K, L3K, G4K, P5K, A6K, S7K, S8K, L9K, P10K, Q11K, S12K, F13K, L14K, L15K, E19K, Q20K, V21K, Q25K, G26K, D27K, A29K, A30K, E33K, A37K, T38K, Y39K, L41K, H43K, P44K, E45K, E46K, V48K, L49K, L50K, H52K, S53K, L54K, 156K, P57K, P60K, L61K, S62K, S63K, P65K, S66K, Q67K, A68K, L69K, Q70K, L71K, A72K, G73K, S76K, Q77K, L78K, S80K, F83K, Q86K, G87K, Q90K, E93K, G94K, S96K, P97K, E98K, L99K, G100K, P101K, T102K, D104K, T105K, Q107K, L108K, D109K, A111K, D112K, F113K, T115K, T116K, W118K, Q119K, Q120K, M121K, E122K, E123K, L124K, M126K, A127K, P128K, A129K, L130K, Q131K, P132K, T133K, Q134K, G135K, A136K, M137K, P138K, A139K, A141K, S142K, A143K, F144K, Q145K, S155K, H156K, Q158K, S159K, L161K, E162K, V163K, S164K, Y165K, V167K, L168K, H170K, L171K, A172K, Q173K and P174K (where residue position is relative to SEQ ID NO: 1).

Examples of preferred amino acid substitutions thus include one or more of Q70K, Q90K, T105K, Q120K, T133K, S159K and H170K/Q/R, such as two, three, four or five of these substitutions, for example: Q70K+Q90K, Q70K+T105K, Q70K+Q120K, Q70K+T133K, Q70K+S159K, Q70K+H170K, Q90K+T105K, Q90K+Q120K, Q90K+T133K, Q90K+S159K, Q90K+H170K, T105K+Q120K, T105K+T133K, T105K+S159K, T105K+H170K, Q120K+T133K, Q120K+S159K, Q120K+H170K, T133K+S159K, T133K+H170K, S159K+H170K, Q70K+Q90K+T105K, Q70K+Q90K+Q120K, Q70K+Q90K+T133K, Q70K+Q90K+S159K, Q70K+Q90K+H170K, Q70K+T105K+Q120K, Q70K+T105K+T133K, Q70K+T105K+S159K, Q70K+T105K+H170K, Q70K+Q120K+T133K, Q70K+Q120K+S159K, Q70K+Q120K+H170K, Q70K+T133K+S159K, Q70K+T133K+H170K, Q70K+S159K+H170K, Q90K+T105K+Q120K, Q90K+T105K+T133K, Q90K+T105K+S159K, Q90K+T105K+H170K, Q90K+Q120K+T133K, Q90K+Q120K+S159K, Q90K+Q120K+H170K, Q90K+T133K+S159K, Q90K+T133K+H170K, Q90+S159K+H170K, T105K+Q120K+T133K, T105K+Q120K+S159K, T105K+Q120K+H170K, T105K+T133K+S159K, T105K+T133K+H170K, T105K+S159K+H170K, Q120K+T133K+S159K, Q120K+T133K+H170K, Q120K+S159K+H170K, T133K+S159K+H170K, Q70K+Q90K+T105K+Q120K, Q70K+Q90K+T105K+T133K, Q70K+Q90K+T105K+S159K, Q70K+Q90K+T105K+H170K, Q70K+Q90K+Q120K+T133K, Q70K+Q90K+Q120K+S159K, Q70K+Q90K+Q120K+H170K, Q70K+Q90K+T133K+S159K, Q70K+Q90K+T133K+H170K, Q70K+Q90K+S159K+H170K, Q70K+T105K+Q120K+T133K, Q70K+T105K+Q120K+S159K, Q70K+T105K+Q120K+H170K, Q70K+T105K+T133K+S159K, Q70K+T105K+T133K+H170K, Q70K+T105K+S159K+H170K, Q70K+Q120K+T133K+S159K, Q70K+Q120K+T133K+H170K, Q70K+T133K+S159K+H170K, Q90K+T105K+Q120K+T133K, Q90K+T105K+Q120K+S159K, Q90K+T105K+Q120K+H170K, Q90K+T105+T133K+S159K, Q90K+T105+T133K+H170K, Q90K+T105+S159K+H170K, Q90K+Q120K+T133K+S159K, Q90K+Q120K+T133K+H170K, Q90K+Q120K+S159K+H170K, Q90K+T133K+S159K+H170K, T105K+Q120K+T133K+S159K, T105K+Q120K+T133K+H170K, T105K+Q120K+S159K+H170K, T105K+T133K+S159K+H170K or Q120K+T133K+S159K+H170K. In any of the variants listed above, the substitution H170K may instead be H170Q or H170R. Particularly preferred substitutions to introduce a lysine include one or both of T105K and S159K.

In a further embodiment, the G-CSF polypeptide may be altered to produce a G-CSF variant in which one or more of the native lysine residues in positions 16, 23, 34 and 40 is removed in order to avoid PEGylation at these positions. For example, one or more of these lysine residues may be removed by way of substitution, preferably with an arginine or glutamine residue, more preferably with an arginine residue. Preferably, one or more of the lysine residues at positions 16, 34 and 40 are removed by way of substitution, more preferably two or three of these lysine are removed, and most preferably all three of the lysines at this position are removed by substitution. Thus, in a preferred embodiment the G-CSF variant comprises the sequence of SEQ ID NO: 1 with at least one substitution selected from the group consisting of K16R, K16Q, K34R, K34Q, K40R and K40Q; that is, at least one substitution selected from the group consisting of K16R/Q, K34R/Q and K40R/Q. In a particularly preferred embodiment, the variant comprises the substitutions K16R/Q+K34R/Q+K40R/Q, such as, for example, K16R+K34R+K40R or K16Q+K34R+K40R or K16R+K34Q+K40R or K16R+K34R+K40Q or K16Q+K34Q+K40R or K16R+K34Q+K40Q or K16Q+K34Q+K40Q.

In another embodiment, the G-CSF variant comprises at least one substitution to introduce a lysine residue together with at least one substitution to remove a lysine residue as explained above.

In another embodiment, the multi-PEGylated G-CSF variant comprises a substitution of one or more of the lysine residues at positions 16, 34, and 40, such as with an arginine or a glutamine residue, e.g., an arginine residue, and one or more substitution selected from Q70K, Q90K, T105K, Q120K, T133K, and S159K, and is conjugated to 2-6, such as 2-4, polyethylene glycol moieties each with a molecular weight of about 1000-10,000 Da.

In another embodiment, the multi-PEGylated G-CSF variant comprises one or more substitution selected from K16R, K34R, and K40R, and one or more substitution selected from Q70K, Q90K, T105K, Q120K, T133K, and S159K, and is conjugated to 2-6, such as 2-4, polyethylene glycol moieties each with a molecular weight of about 1000-10,000 Da.

In another embodiment, the multi-PEGylated G-CSF variant comprises a substitution of one or more of the lysine residues at positions 16, 34, and 40, such as with an arginine or a glutamine residue, e.g., an arginine residue, and at least one substitution selected from T105K and S159K, and is conjugated to 2-6, such as 2-4, polyethylene glycol moieties each with a molecular weight of about 1000-10,000 Da.

In another embodiment, the multi-PEGylated G-CSF variant comprises one or more substitution selected from K16R, K34R, and K40R, and at least one substitution selected from T105K and S159K, and is conjugated to 2-6, such as 2-4, polyethylene glycol moieties each with a molecular weight of about 1000-10,000 Da.

In a particular embodiment the multi-PEGylated G-CSF variant comprises the substitutions K16R, K34R, K40R, T105K and S159K and is conjugated to 2-6, such as 2-4, polyethylene glycol moieties with a molecular weight of about 1000-10,000 Da.

In a particular embodiment, the multi-PEGylated G-CSF variant may have 2-6, typically 2-5, such as 2-4, polyethylene glycol moieties with a molecular weight of about 5000-6000 Da attached, e.g. mPEG with a molecular weight of about 5 kDa. Preferably, the multi-PEGylated G-CSF variant has 2-4 polyethylene glycol moieties with a molecular weight of about 5000-6000 Da attached, e.g. 5 kDa mPEG. A particularly preferred multi-PEGylated G-CSF variant that is suitable for use in the method of the invention comprises the substitutions K16R, K34R, K40R, T105K and S159K and contains 2-4 PEG moieties each with a molecular weight of about 5 kDa, such as 3 such PEG moieties.

In another embodiment, the multi-PEGylated G-CSF variant may be produced so as to have only a single number of PEG moieties attached, e.g. either 2, 3, 4 or 5 PEG moieties per conjugate, or to have a desired mix of conjugates with different numbers of PEG moieties attached, e.g. a mix of conjugates having 2-5, 2-4, 3-5, 3-4, 4-6, 4-5 or 5-6 attached PEG moieties. As indicated above, an example of a preferred conjugate mix is one having 2-4 PEG moieties of about 5 kDa, for example a conjugate having primarily 3 PEG moieties attached per conjugate but with a small proportion of the conjugates having either 2 or 4 PEG moieties attached.

It will be understood that a conjugate having a specific number of attached PEG moieties, or a mix of conjugates having a defined range of numbers of attached PEG moieties, may be obtained by choosing suitable PEGylation conditions and optionally by using subsequent purification to separate conjugates having the desired number of PEG moieties. Examples of methods for separation of G-CSF conjugates with different numbers of PEG moieties attached as well as methods for determining the number of PEG moieties attached are described, e.g. in WO 01/51510 and WO 03/006501, both of which are incorporated herein by reference. For purposes of the present invention, a conjugate may be considered to have a given number of attached PEG moieties if separation on an SDS-PAGE gel shows no or only insignificant bands other than the band(s) corresponding to the given number(s) of PEG moieties. For example, a sample of a conjugate is considered to have 3 attached PEG groups if an SDS-PAGE gel on which the sample has been run shows a major bands corresponding to 3 PEG groups, respectively, and only insignificant bands or, preferably, no bands corresponding to 2 or 4 PEG groups.

In some cases, amine-specific activated PEG derivatives such as mPEG-SPA may not attach exclusively to the N-terminus and the ε-amino groups of lysine residues via an amide bond, but may also attach to the hydroxy group of a serine, tyrosine or threonine residue via an ester bond. As a result, the PEGylated proteins may not have a sufficient degree of uniformity and may contain a number of different PEG isomers other than those that were intended. Such PEG moieties bound via an ester bond will typically be labile and can be removed by the method described in U.S. Provisional Patent Application No. 60/686,726, incorporated herein by reference, which involves subjecting the PEGylated polypeptide to an elevated pH for a period of time sufficient to remove the labile PEG moieties attached to a hydroxy group. This method is also described in U.S. Ser. No. 11/420,546 (U.S. Pat. No. 7,381,805) and WO 2006/128460, each of which are incorporated herein by reference.

In a preferred embodiment, the multi-PEGylated G-CSF variant is a mixture of positional PEG isomer species. As used herein, the term “positional PEG isomer” of a protein refers to different PEGylated forms of the protein where PEG groups are located at different amino acid positions of the protein. A preferred multi-PEGylated G-CSF variant employed in the practice of the present invention is a mixture of lysine/N-terminal PEG isomers. The term “lysine/N-terminal PEG isomer” of a protein means that the PEG groups are attached to the amino-terminal of the protein and/or to epsilon amino groups of lysine residues in the protein. For example, the phrase “lysine/N-terminal positional PEG isomers having 3 attached PEG moieties”, as applied to G-CSF, means a mixture of G-CSF positional PEG isomers in which three PEG groups are attached to epsilon amino groups of lysine residues and/or to the N-terminus of the protein. Typically, a “lysine/N-terminal positional PEG isomer having 3 attached PEG moieties” will have two PEG moieties attached to lysine residues and one PEG moiety attached to the N-terminus. Analysis of the positional PEG isomers may be performed using cation exchange HPLC as described in WO 2006/128460, which is incorporated herein by reference.

Typically, the mixture of positional PEG isomer species is a substantially purified mixture of lysine/N-terminal positional PEG isomers. A “substantially purified mixture of lysine/N-terminal positional PEG isomers” of a polypeptide refers to a mixture of lysine/N-terminal positional PEG isomers which has been subjected to a chromatographic or other purification procedure in order to remove impurities such as non-lysine/N-terminal positional PEG isomers. The “substantially purified mixture of lysine/N-terminal positional PEG isomers” will, for example, be free of most labile PEG moieties attached to a hydroxyl group that would otherwise be present in the absence of a partial de-PEGylation step and subsequent purification as described herein, and will typically contain less than about 20% polypeptides containing a labile PEG moiety attached to a hydroxyl group, more typically less than about 15%. Preferably, there will be less than about 10% polypeptides containing a labile PEG moiety attached to a hydroxyl group, for example, less than about 5%.

Preferably, the mixture of positional PEG isomer species is a homogeneous mixture of positional PEG isomers of a G-CSF variant. The term “homogeneous mixture of positional PEG isomers of a polypeptide (G-CSF) variant” means that the polypeptide moiety of the different positional PEG isomers is the same. This means that the different positional PEG isomers of the mixture are all based on a single polypeptide variant sequence. For example, a homogeneous mixture of positional PEG isomers of a PEGylated G-CSF polypeptide variant means that different positional PEG isomers of the mixture are based on a single G-CSF polypeptide variant.

Typically, the homogeneous mixture of positional PEG isomers of a G-CSF variant exhibits substantial uniformity. As used herein, “uniformity” refers to the homogeneity of a PEGylated polypeptide in terms of the number of different positional isomers, i.e., different polypeptide isomers containing different numbers of PEG moieties attached at different positions, as well as the relative distribution of these positional isomers. For pharmaceutical polypeptides intended for therapeutic use in humans or animals, it is generally desirable that the number of different positional PEG isomers and different PEGylated species is minimized.

In one embodiment (referred to as “Maxy-G21” in the examples hereinbelow), the multi-PEGylated G-CSF variant is a mixture of positional PEG isomers where the G-CSF variant component has the amino acid sequence of SEQ ID NO:1 with the substitutions K16R, K34R, K40R, T105K and S159K (relative to SEQ ID NO:1), comprising positional isomers each having either 4 or 5 attached PEG moieties, including labile PEG moieties at one or both of Ser66 or Tyr165, as well as stable PEG moieties at the N-terminus and at one or two of positions K23, K105 and K159. The multi-PEGylated G-CSF variant referred to as Maxy-G21 herein comprises PEG moieties that are mPEG-SPA (Nektar), each having an average molecular weight of 5000 Da.

The term, “partial de-PEGylation” refers herein to the removal of labile PEG moieties attached to a hydroxyl group, while PEG moieties that are more stably attached to the N-terminal or the amino group of a lysine residue remain intact. The method for carrying out this process is described in U.S. Ser. No. 60/686,726, U.S. Ser. No. 11/420,546 (U.S. Pat. No. 7,381,805), and WO 2006/128460, each of which are incorporated herein by reference.

In another embodiment (referred to as “Maxy-G34” in the examples hereinbelow), the multi-PEGylated G-CSF variant is a mixture of positional PEG isomers where the G-CSF variant component has the amino acid sequence of SEQ ID NO:1 with the substitutions K16R, K34R, K40R, T105K and S159K (relative to SEQ ID NO:1), and where at least 80% of the mixture contains 2 species of positional PEG isomers each having 3 attached PEG moieties, where one of the isomers has PEG groups attached at the N-terminal, Lys 23 and Lys 159 and the other isomer has PEG groups attached at the N-terminal, Lys 105 and Lys 159. The multi-PEGylated G-CSF variant referred to as Maxy-G34 herein comprises PEG moieties that are mPEG-SPA (Nektar), each having an average molecular weight of 5000 Da.

For all the embodiments described above, the G-CSF variant and the multi-PEGylated G-CSF variant may optionally include a methionine residue added to the N-terminus.

In further embodiments, the multi-PEGylated G-CSF variant to be administered according to the invention may be prepared as described in any of the following, each of which are incorporated herein by reference:
WO 89/05824 (lysine-depleted variants of G-CSF)
U.S. Pat. No. 5,824,778 (G-CSF having at least one PEG molecule covalently attached to at least one amino acid of the polypeptide through a carboxyl group of said amino acid)
WO 99/03887 (PEGylated cysteine variants of G-CSF)
WO 2005/055946 (“glyco-PEGylated” G-CSF conjugates with PEG moieties linked via an intact glycosyl linking group)
WO 2005/070138 (G-CSF polypeptides comprising a mutant peptide sequence encoding an O-linked glycosylation site that does not exist in the corresponding wild-type polypeptide).
US 2005/0114037 A1 (G-CSF with at least one polymeric moiety attached at least one of a number of different specified amino acid positions)

In another embodiment, the multi-PEGylated G-CSF variant to be administered according to the invention exhibits an improved pharmacokinetic property, such as an increased serum half-life and/or an increased AUC, compared to the mono-PEGylated hG-CSF, Neulasta®. Preferably, the multi-PEGylated G-CSF variant exhibits a serum half-life or an AUC increased by at least about 1.2× of the serum half-life or AUC of Neulasta®, e.g. increased by at least about 1.4×, such as by at least about 1.5×, e.g. by at least about 1.6×, such as by at least about 1.8×, e.g. by at least about 2.0×, 2.5×, 3×, 5×, or 10× that of the mono-PEGylated hG-CSF, Neulasta®.

### Radiation Exposure and Treatment

### A. Effects of Radiation Exposure on the Hematopoietic System.

Radiation accident scenarios have provided several defining characteristics useful in the design of emergency preparedness models and treatment strategies for severely-irradiated individuals. Body position, fortuitous shielding and distance relative to the source will result in unilateral, non-uniform and heterogeneous exposures to any group of individuals. Additionally, the time interval between exposure and initiation of treatment may be less than optimal. These exposure aspects underscore the difficulty in determining an accurate absorbed dose; the basis for establishing triage and treatment and furthermore, the effect of treatment on biodosimetry is unknown. Regarding the radiation exposure it is reasonable to assume the above characteristics forecast a highly variable dose distribution, with possible sparing of bone marrow-derived hematopoietic stem and progenitor cells (“BM-derived HSC and HPC”) and thymic tissue, thereby enhancing the potential for hematopoietic and lymphoid regeneration in response to timely administration of hematopoietic growth factors (“HGF”).

The hematopoietic system is the most radiosensitive and the dose-limiting organ system following acute total body irradiation (TBI). HSC and HPC are killed in a dose-dependent exponential fashion with minimal repair capacity, dictating that modest increases in exposure dose results in disproportionately increased death of HSC and HPC. Mature, more differentiated cells are more radioresistant than the highly proliferative stem and progenitor cells. It has been proposed that a subset of HSC is relatively radioresistant. The exponential, dose-dependent nature of cell kill for HSC and HPC in concert with the reality of non-uniform radiation exposure and consequent variable dose distribution across the active bone marrow suggests that a small or modest fraction of HSC and HPC, as well as cells of the respective BM (osteoblast), vascular and thymic (epithelial cell) niches will survive potentially lethal doses of radiation in the hematopoietic syndrome and be amenable to the therapeutic approaches as outlined herein.

Acute exposure resulting from a nuclear explosion or accident will likely be unilateral, non uniform and with some degree of partial body shielding. Consequently a fraction of HSC and HPC located within the marrow and vascular niches may not be exposed, or exposed only to a significantly lower dose of radiation. There is a consistent data base in animal models demonstrating the sparing effect of partial-body or non-uniform irradiation. Unilateral exposure can result in an approximate 20% increase in LD50/30 values (the average dose of radiation which results in death of 50% of the subjects within 30 days) for unilateral versus bilateral exposure. The orientation to the radiation source must also be assessed in biological terms. Dorsal exposure maximizes bone marrow damage, due to the large percent of active bone marrow in the spine and dorsal aspects of ribs and pelvis of young adults. Conversely, ventral exposure minimizes bone marrow damage due to ventral shielding of the bulk of active bone marrow. The non uniform exposure should not be viewed as effective as partial body shielding of bone marrow. This is significant because of the exponential relationship between radiation dose and HSC/HPC survival, e.g., halving the total body dose does not increase HSC survival to 50%, but only to 10%.

### B. Radiation Doses

The data base for acute, radiation-induced hematopoietic syndrome in non-human primates (“NHP”) was derived from experiments involving total body irradiation (TBI) with 250 kilovolt peak (kVp) X-radiation or Co-60 gamma and 2 megavolts (MV) X-radiation. The data base for Co-60 gamma radiation-induced lethality is a single, nonpublished experiment (n=90 NHP) performed in 1967. Dalrymple et al. (Radiation Res. 25:377-400, 1965) used 2 MV X-radiation to establish the dose-response relationship for TBI and hematopoietic syndrome lethality. These two studies serve as the basis for establishing the dose-response relationship of radiation-induced hematopoietic syndrome lethality in NHPs exposed to gamma radiation or high energy X-ray (2 MV) that have not received supportive care. This data base has served as the control cohort from which a single dose of radiation and associated lethality could be chosen with a degree of certainty. The LD50/30 values for NHPs obtained from these earlier studies were 6.40 Gy [6.06, 7.75] and 6.65 Gy [6.00, 10.17] (95% confidence interval (CI) in brackets [ ]), respectively. For comparison, the respective LD50/30 value for NHP exposed to TBI with 250 kVp X-rays is approximately 4.80 Gy demonstrating the relative biologic effect of X-irradiation with lower energy X-rays that the 2 MV X-rays used in the Dalrymple experiment.

Data regarding the effects of whole-body or significant partial-body irradiation in humans has necessarily been gleaned from past nuclear incidents, such as the Hiroshima explosion and the Chemobyl accident. Such data is maintained in a registry at the Radiation Emergency Assistance Center/Training Site (REAC/TS) in Oak Ridge, Tenn. Based on this data, since absolute lymphocyte count (ALC) drops soon after exposure to penetrating radiation, a method has been developed to estimate radiation dose in an individual by determining the rate of decrease in lymphocyte count over a 48-hour period (Goans R. E., et al. Health Phys. 81:446-449, 2001). Such estimates require two or more ALC determinations spaced at 4- to 6-hr intervals. In instances where such measurements are impractical, such as in mass casualty situations, another estimate of radiation dose is based on the length of time after radiation exposure before the subject vomits. Berger, M. E. et al. (Occupational Medicine 56:162-172, 2006) provides a table showing that most individuals (70-90%) exposed to acute whole body irradiation of at least 2 Gy will vomit within 1 to 2 hrs after exposure, while essentially 100% of the individuals exposed to at least 4 Gy of radiation will vomit within one hour, and those exposed to at least 6 Gy of radiation will vomit within 30 minutes. The severity and time to onset of other physical symptoms associated with acute, whole-body radiation exposure (such as body temperature, headache, diarrhea) is also tabulated in Berger et al., (supra).

### C. Supportive Care

The use of antibiotics, fluids, blood products, analgesics and nutrition is the “standard of care” for patients exposed to myelosuppressive and lethal doses of radiation. Supportive care alone, such as antibiotics, whole blood or platelet transfusions, fluids and nutrition can significantly enhance the survival of irradiated subjects. The relationship between supportive care and hematopoietic syndrome survival in animals exposed to lethal doses of radiation has been demonstrated in canines, but not in non-human primates (NHPs). A single study by Byron et al demonstrated the ability of an antibiotic regimen alone to significantly increase survival to 72% in rhesus macaques exposed to a 100% lethal dose. Additionally, the MacVittie/Farese laboratories at the Armed Forces Radiobiology Research Institute (AFRRI) and University of Maryland at Baltimore (UMB) established the effect of supportive care at a single lethal dose of TBI (LD70/30) estimated from the data bases noted below. These data show that irradiating NHP with TBI from Co-60 gamma radiation at a dose equivalent to an LD70/30 (i.e., a dose which results in death of 70% of the subjects in 30 days in the absence of supportive care) decreases the number of deaths to approximately 14% of the subjects over 30 days (i.e., LD 14/30) when supportive care is administered. Similar studies were performed (MacVittie/Farese UMB lab) with rhesus macaques exposed to TBI with 250 kVp X-rays. The estimated 70% lethality associated with 6.00 Gy TBI was reduced to 9% with addition of supportive care alone.

The results obtained in the dose-response studies of radiation-induced hematopoietic syndrome lethality in NHPs that have not received supportive care, as described above, were used to design a recent blinded, radiation dose-randomized study to determine the lethal dose response relationship in NHPs receiving supportive care (Example 1). The resultant value for the LD50/60 was 7.52 Gy relative to an approximate 6.50 Gy LD50/60 for the unsupported, historical control cohorts. This served to confirm the survival-enhancing effect of supportive care as well as provide the dose relationship for determining respective LD30/60, LD50/60 and LD70/60 doses for NHP exposed to lethal doses of radiation administered supportive care, otherwise known as medical management within the hematopoietic syndrome.

This survival-enhancing effect is dependent on two conditions. First, the surviving HSC and HPC must be capable of spontaneous regeneration and second, the hematopoietic recovery must result in the production of functional neutrophils and/or platelets within a critical, clinically manageable period of time.

### D. Role of Hematopoietic Growth Factors in the Treatment of ARS

There is a substantial and consistent data base in small and large animal models of myelosuppressive and/or lethal radiation exposure which demonstrate that hematopoietic growth factors (HGFs), when administered at their optimal schedule and in combination with supportive care, significantly enhance survival and recovery of neutrophils and platelets beyond that noted for supportive care alone. The MacVittie laboratory previously established the utility of supportive care alone, as well as in conjunction with administration of G-CSF in dogs exposed to Co-60 TBI at levels which induce the complete hematopoietic syndrome. The LD50/30 with no supportive care was 2.60 Gy, which increased to 3.38 Gy with supportive care and further increased to 4.88 Gy with the addition of G-CSF under its optimum administration schedule. This study used standard laboratory models of irradiation involving uniform TBI at moderate dose-rates.

The conventional schedule for administration of HGFs is to initiate treatment early, within 24 hrs following irradiation, and to continue daily administration to ensure regeneration of hematopoietic progenitor cells and production of neutrophils and/or platelets. However, a more realistic schedule with regard to treatment following a nuclear explosion or accident is the delayed administration for 48-72 hrs post irradiation. A number of preclinical studies have been performed assessing the effect of delayed administration of HGFs. The majority of these studies show that the magnitude of the hematopoietic response was significantly lessened by an increased time interval between HGF administration and irradiation. Along with G-CSF and PEGylated G-CSF, other HGFs sometimes used in treatment of ARS include granulocyte macrophage colony stimulating factor (GM-CSF), stem cell factor (SCF), FLT3-ligand (FL), interleukin-3 (IL-3), megakaryocyte growth and development factor (MGDF), thrombopoietin (TPO), TPO-receptor agonist, and erythropoietin (EPO) (Drouet, M. et al., Haematologica 93(3)465-466, 2008; Herodin F. et al., Experimental Hematology 35:1172-1181, 2007). Of these, as single agents, only G-CSF and GM-CSF are currently available for treating potentially lethally-irradiated personnel, if used “off-label”. These HGFs would likely be the first proposed to the FDA for approval under the FDA “Animal Rule” (AR). Consideration of HGF “cocktails” must include analysis of respective toxicities and administration time post exposure.

### E. Multi-PEGylated G-CSF Variants in the Treatment of Radiation-Induced Neutropenia in Animal Model Systems

Radiation-induced cytopenia in the rhesus monkey has proven to be an effective model system for studying the efficacy of pharmaceuticals in treating thrombocytopenia and neutropenia. In the study described in Example 2, a single injection of an exemplary multi-PEGylated G-CSF variant according to the invention (identified herein as “Maxy-G21”) induced a significant increase in peripheral blood total nucleated cells, neutrophils, mononuclear cells and a significant mobilization of colony-forming cells into the peripheral blood. Compared to control animals exposed to 6.0 Gy TBI, which exhibited a period of neutropenia of 14.8±15.2 days, animals administered Maxy-G21 at a dose 300 μg/kg at 24 hours following TBI exhibited a significantly shortened period of neutropenia of 7.3±1.1 days. The duration of neutropenia was determined as the number of days that the animal had an observed or an imputed ANC below 500/μL. The ANC nadir, defined as the first lowest observed or imputed ANC that occurred at least 2 days after the first dose of the test compound, was also markedly improved to 140±45/μL to from 49±22/μL in control animals. The time to recovery determined as the number of days from study day 1 until the first 2 consecutive days with observed or imputed ANC of 500/μL or above, was likewise improved from a control value of 21.2±0.4 days to 15.5±0.3 days in the Maxy-G21 treated cohort.

As compared to a combined Neulasta® cohort, comprising the intra-study Neulasta® group and a historical cohort (n=9), Maxy-G21 significantly shortened the duration of neutropenia (p=0.02) as well as time to recovery. The antibiotic requirements were also significantly different from the Neulasta® group, as the Maxy-G21 treated cohort only required antibiotics for 9.8 days where as the combined Neulasta®-treated cohort required 14.7 days of antibiotic support.

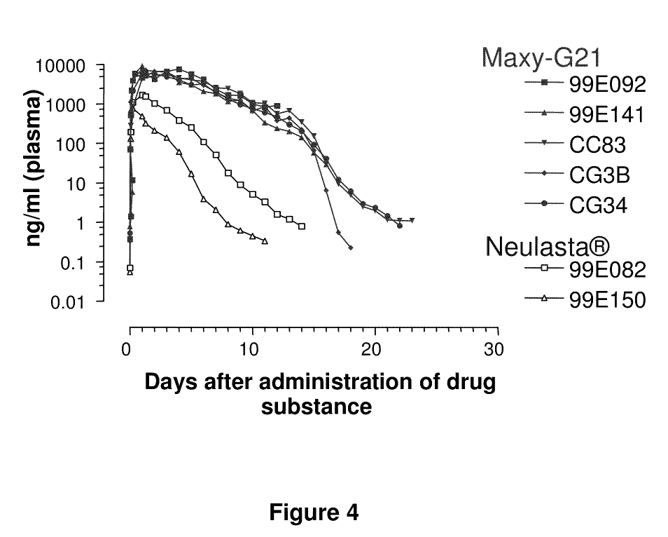

The study described in Example 2 demonstrated that an exemplary multi-PEGylated G-CSF variant according to the invention (identified herein as Maxy-G21) administered s.c. to rhesus monkeys significantly shortened the period of neutropenia in irradiated NHP. The effect was furthermore found to exceed that of Neulasta® when compared to a cohort comprising the intra-study Neulasta® cohort and a historical Neulasta® cohort (N=9). The pharmacokinetic data provided evidence that the multi-PEGylated G-CSF variant exhibits a markedly extended plasma half-life as compared to Neulasta® in irradiated macaques (FIG. 4). The PK data thus support the working hypothesis that a multi-PEGylated G-CSF variant has a greater bioavailability than the mono-PEGylated hG-CSF, Neulasta®, both in NHP undergoing a state of severe radiation-induced myelosuppression, as well as in healthy (non-irradiated) NHP.

Overall, the multi-PEGylated G-CSF variant was found to markedly shorten the period of radiation-induced neutropenia in non-human primates. The reduction of the period of neutropenia was furthermore found to exceed that of Neulasta® when compared to a historical Neulasta® cohort. The extent and duration of radiation-induced neutropenia was significantly diminished by the administration of a multi-PEGylated G-CSF variant in accordance with the methods of the present invention.

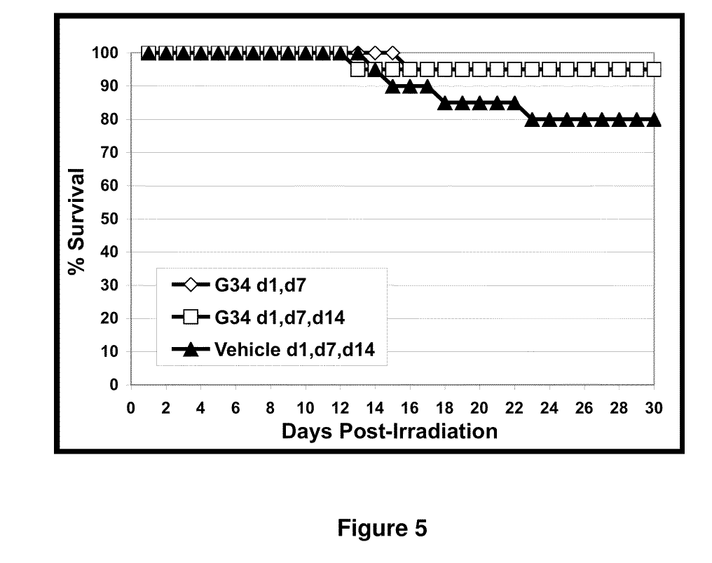

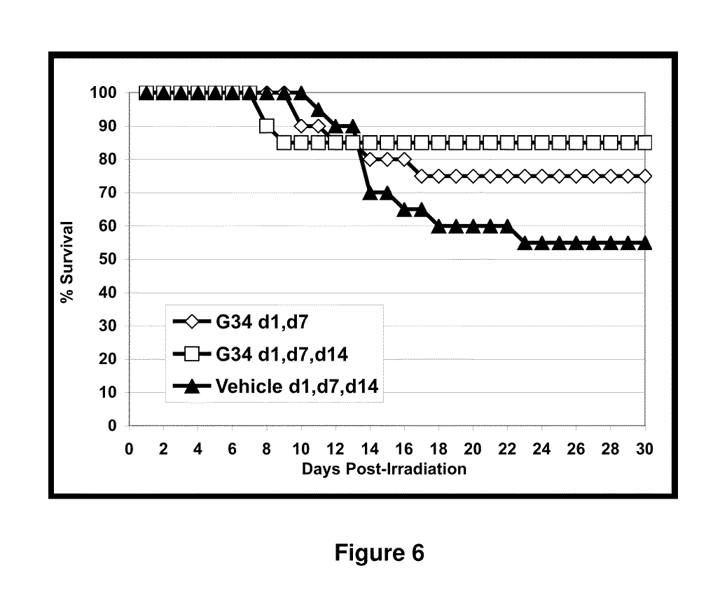

In the study described in Example 3, mice were exposed to doses of radiation sufficient to kill either 20% of the untreated control animals (7.76 Gy; LD20/30) or 45% of the untreated control animals (7.96 Gy; LD45/30). On day one after TBI, the animals were administered either an exemplary multi-PEGylated G-CSF variant according to the invention (identified herein as “Maxy-G34”) at a dosage of 20 μg/20 g mouse, or diluent. The dosage was repeated on day 7 and, in some animals, on day 14. Mice administered the multi-PEGylated G-CSF variant after irradiation at the LD20/30 level and the LD45/30 level exhibited significantly greater percentage of survival after 30 days compared to the untreated animals (FIGS. 5 and 6, respectively).

The studies presented in Examples 2 and 3 demonstrate that multi-PEGylated G-CSF variants according to the invention are effective at reducing the extent and duration of radiation-induced neutropenia and extending survival in two animal model systems. Multi-PEGylated G-CSF variants may thus be effective in the treatment of neutropenia associated with life-threatening radiation exposure, as in the ARS in the event of a nuclear emergency.

### Administration of Multi-PEGylated G-CSF Variant

### A. Dosages

The dosage of the multi-PEGylated G-CSF variant administered according to the invention will generally be a similar order of magnitude as the current approved dosage for mono-PEGylated hG-CSF (Neulasta®) in chemotherapeutic applications, which is 6 mg per adult human patient (e.g., 100 μg/kg for a 60 kg patient). An appropriate dose of the multi-PEGylated G-CSF variant is therefore contemplated to be in the range of from about 1 mg to about 30 mg, such as from about 2 mg to about 20 mg, e.g. from about 3 mg to about 15 mg. A suitable dose may thus be, for example, about 1 mg, about 2 mg, about 3 mg, about 6 mg, about 9 mg, about 12 mg, about 15 mg, about 20 mg, or about 30 mg. Alternatively, dosage may be based on the weight of the patient, such that an appropriate dose of the multi-PEGylated G-CSF variant is contemplated to be in the range of from about 20 μg/kg to about 500 μg/kg, such as about 30 μg/kg to about 400 μg/kg, such as about 40 μg/kg to about 300 μg/kg, e.g. from about 50 μg/kg to about 200 μg/kg. A suitable dose may thus be, for example, about 20 μg/kg, about 30 μg/kg, about 40 μg/kg, about 50 μg/kg, about 60 μg/kg, about 75 μg/kg, about 100 μg/kg, about 125 μg/kg, about 150 μg/kg, about 175 μg/kg, about 200 μg/kg, about 250 μg/kg, about 300 μg/kg, about 400 μg/kg, or about 500 μg/kg. The multi-PEGylated G-CSF variant is preferably administered as soon as possible following radiation exposure, e.g., within seven days, within four days, within three days, within two days (i.e., within 48 hours) or more preferably within one day (i.e., within 24 hours) following radiation exposure. Depending on the nature of the illness and the prognosis and response of the patient, a second and possibly third administration of multi-PEGylated G-CSF variant may be given between one to four weeks (e.g., about 7 days, about 10 days, about 14 days, about 18 days, about 21 days, about 24 days, about 28 days) after the prior administration.

The precise dosage and frequency of administration of the multi-PEGylated G-CSF variant will depend on a number of factors, such as the specific activity and the pharmacokinetic properties of the multi-PEGylated G-CSF variant, as well as the nature and the severity of the condition being treated (such as, the level and/or duration of the radiation exposure, the area and amount of body exposed, the type of radiation, the severity of the ARS-associated symptoms), among other factors known to those of skill in the art. Normally, the dose should be capable of preventing or lessening the extent and/or duration of neutropenia in the subject. Such a dose may be termed an “effective” or “therapeutically effective” amount. It will be apparent to those of skill in the art that an effective amount of the multi-PEGylated G-CSF variant of the invention depends, inter alia, upon the severity of the condition being treated, the dose, the administration schedule, whether the multi-PEGylated G-CSF variant is administered alone or in combination with other therapeutic agents, the serum half-life and other pharmacokinetic properties of the multi-PEGylated G-CSF variant, as well as the size, age, and general health of the patient. The dosage and frequency of administration is ascertainable by one skilled in the art using known techniques.

### B. Pharmaceutical Compositions

The multi-PEGylated G-CSF variant administered according to the present invention may be administered in a composition including one or more pharmaceutically acceptable carriers or excipients. The multi-PEGylated G-CSF variant can be formulated into pharmaceutical compositions in a manner known per se in the art to result in a pharmaceutical that is sufficiently storage-stable and is suitable for administration to humans or animals. The pharmaceutical composition may be formulated in a variety of forms, including as a liquid or gel, or lyophilized, or any other suitable form. The preferred form will depend upon the particular indication being treated and will be apparent to one of skill in the art.

“Pharmaceutically acceptable” means a carrier or excipient that at the dosages and concentrations employed does not cause any untoward effects in the patients to whom it is administered. Such pharmaceutically acceptable carriers and excipients are well known in the art (see, e.g., Remington's Pharmaceutical Sciences, 18th edition, A. R. Gennaro, Ed., Mack Publishing Company (1990); Pharmaceutical Formulation Development of Peptides and Proteins, S. Frokjaer and L. Hovgaard, Eds., Taylor & Francis (2000); and Handbook of Pharmaceutical Excipients, 3rd edition, A. Kibbe, Ed., Pharmaceutical Press (2000)).

### C. Parenteral Compositions

An example of a pharmaceutical composition is a solution designed for parenteral administration, e.g. by the subcutaneous route. Although in many cases pharmaceutical solution formulations are provided in liquid form, appropriate for immediate use, such parenteral formulations may also be provided in frozen or in lyophilized form. In the former case, the composition must be thawed prior to use. The latter form is often used to enhance the stability of the active compound contained in the composition under a wider variety of storage conditions, as it is recognized by those skilled in the art that lyophilized preparations are generally more stable than their liquid counterparts. Such lyophilized preparations are reconstituted prior to use by the addition of one or more suitable pharmaceutically acceptable diluents such as sterile water for injection or sterile physiological saline solution.

In case of parenterals, they are prepared for storage as lyophilized formulations or aqueous solutions by mixing, as appropriate, the polypeptide having the desired degree of purity with one or more pharmaceutically acceptable carriers, excipients or stabilizers typically employed in the art (all of which are termed “excipients”), for example buffering agents, stabilizing agents, preservatives, isotonifiers, non-ionic detergents, antioxidants and/or other miscellaneous additives.

Buffering agents help to maintain the pH in the range which approximates physiological conditions. They are typically present at a concentration ranging from about 2 mM to about 50 mM Suitable buffering agents for use with the present invention include both organic and inorganic acids and salts thereof such as citrate buffers (e.g., monosodium citrate-disodium citrate mixture, citric acid-trisodium citrate mixture, citric acid-monosodium citrate mixture, etc.), succinate buffers (e.g., succinic acid-monosodium succinate mixture, succinic acid-sodium hydroxide mixture, succinic acid-disodium succinate mixture, etc.), tartrate buffers (e.g., tartaric acid-sodium tartrate mixture, tartaric acid-potassium tartrate mixture, tartaric acid-sodium hydroxide mixture, etc.), fumarate buffers (e.g., fumaric acid-monosodium fumarate mixture, fumaric acid-disodium fumarate mixture, monosodium fumarate-disodium fumarate mixture, etc.), gluconate buffers (e.g., gluconic acid-sodium glyconate mixture, gluconic acid-sodium hydroxide mixture, gluconic acid-potassium glyuconate mixture, etc.), oxalate buffer (e.g., oxalic acid-sodium oxalate mixture, oxalic acid-sodium hydroxide mixture, oxalic acid-potassium oxalate mixture, etc.), lactate buffers (e.g., lactic acid-sodium lactate mixture, lactic acid-sodium hydroxide mixture, lactic acid-potassium lactate mixture, etc.) and acetate buffers (e.g., acetic acid-sodium acetate mixture, acetic acid-sodium hydroxide mixture, etc.). Additional possibilities are phosphate buffers, histidine buffers and trimethylamine salts such as Tris.

Preservatives are added to retard microbial growth, and are typically added in amounts of about 0.2%-1% (w/v). Suitable preservatives for use with the present invention include phenol, benzyl alcohol, meta-cresol, methyl paraben, propyl paraben, octadecyldimethylbenzyl ammonium chloride, benzalkonium halides (e.g. benzalkonium chloride, bromide or iodide), hexamethonium chloride, alkyl parabens such as methyl or propyl paraben, catechol, resorcinol, cyclohexanol and 3-pentanol.

Isotonicifiers are added to ensure isotonicity of liquid compositions and include polyhydric sugar alcohols, preferably trihydric or higher sugar alcohols, such as glycerin, erythritol, arabitol, xylitol, sorbitol and mannitol. Polyhydric alcohols can be present in an amount between 0.1% and 25% by weight, typically 1% to 5%, taking into account the relative amounts of the other ingredients.

Stabilizers refer to a broad category of excipients which can range in function from a bulking agent to an additive which solubilizes the therapeutic agent or helps to prevent denaturation or adherence to the container wall. Typical stabilizers can be polyhydric sugar alcohols (enumerated above); amino acids such as arginine, lysine, glycine, glutamine, asparagine, histidine, alanine, ornithine, L-leucine, 2-phenylalanine, glutamic acid, threonine, etc., organic sugars or sugar alcohols, such as lactose, trehalose, stachyose, mannitol, sorbitol, xylitol, ribitol, myoinisitol, galactitol, glycerol and the like, including cyclitols such as inositol; polyethylene glycol; amino acid polymers; sulfur-containing reducing agents, such as urea, glutathione, thioctic acid, sodium thioglycolate, thioglycerol, α-monothioglycerol and sodium thiosulfate; low molecular weight polypeptides (i.e. <10 residues); proteins such as human serum albumin, bovine serum albumin, gelatin or immunoglobulins; hydrophilic polymers such as polyvinylpyrrolidone; monosaccharides such as xylose, mannose, fructose and glucose; disaccharides such as lactose, maltose and sucrose; trisaccharides such as raffinose, and polysaccharides such as dextran. Stabilizers are typically present in the range of from 0.1 to 10,000 parts by weight based on the active protein weight.

Non-ionic surfactants or detergents (also known as “wetting agents”) may be present to help solubilize the therapeutic agent as well as to protect the therapeutic polypeptide against agitation-induced aggregation, which also permits the formulation to be exposed to shear surface stress without causing denaturation of the polypeptide. Suitable non-ionic surfactants include polysorbates (20, 80, etc.), polyoxamers (184, 188 etc.), Pluronic® polyols, polyoxyethylene sorbitan monoethers (Tween®-20, Tween®-80, etc.).

Additional miscellaneous excipients include bulking agents or fillers (e.g. starch), chelating agents (e.g. EDTA), antioxidants (e.g., ascorbic acid, methionine, vitamin E) and cosolvents.

The active ingredient may also be entrapped in microcapsules prepared, for example, by coascervation techniques or by interfacial polymerization, for example hydroxymethylcellulose, gelatin or poly-(methylmethacylate) microcapsules, in colloidal drug delivery systems (for example liposomes, albumin microspheres, microemulsions, nano-particles and nanocapsules) or in macroemulsions. Such techniques are disclosed in Remington's Pharmaceutical Sciences, supra.

Parenteral formulations to be used for in vivo administration must be sterile. This is readily accomplished, for example, by filtration through sterile filtration membranes.

The invention is further described by the following non-limiting examples.

### EXAMPLES

### Example 1

### Lethal Radiation Dose Response and the Effect of Supportive Care in a Non-Human Primate Model of Radiation-Induced Neutropenia

The following describes a pilot study designed to define the dose response in rhesus macaques exposed to increasing doses of total body ionizing radiation (TBI) and receiving supportive care (also termed “medical management”). This study was designed to assess:

1. The LD50/30 and supporting radiation-dose survival curves for rhesus macaques exposed to lethal doses of TBI with LINAC-derived 6 MV (average energy, 2 MV) photons plus medical management, and

2. The effect of medical management on the respective LD50/30 and dose response relationship for TBI alone compared to historical data sets.

### Materials and Methods

Forty eight (48) male rhesus monkeys were exposed to bilateral, uniform, total body irradiation (TBI) using a 6 megavolt (MV) LINAC photon source (Varian model #EX-21) (average 2 MV photons) at a dose rate of 80±2.5 cGy/min. Animals in groups of 2-8 per radiation dose were irradiated at six randomized doses of TBI: 7.20 Gy, 7.55 Gy, 7.85 Gy, 8.05 Gy, 8.40 Gy, and 8.90 Gy. Medical management was provided consisting of antibiotics, fluids, blood transfusions, nutritional support, anti-diarrheals, anti-ulceratives, antipyretics and pain management. Irradiated animals were observed for 60 days post TBI.

The primary clinically relevant parameter was 60 day mortality. Secondary endpoints were key neutrophil- and platelet (PLT)-related parameters including: respective neutrophil and platelet nadirs, duration of neutropenia (ANC <500/μl) and thrombocytopenia (PLT <20,000/μl), and time to recovery to an ANC >1,000/μl and PLT >20,000/μl. The day of and duration of ANC <100/μl was also recorded. Other parameters included the number of days with fever (Temp ≧103° F.), incidence of documented infection, febrile neutropenia and mean survival time (MST) of decedents.

Data were collected for 60 days on 48 male rhesus macaques exposed to TBI in 6 dose groups of 8 animals each, at 7.20, 7.55, 7.85, 8.05, 8.40 and 8.90 Gy. Mortality rates were calculated for each dose group.

Descriptive analysis and logistic regression were performed using SAS version 9 and LD estimation was performed using SPLUS version 6.2. Logistic regression analysis was conducted as two-sided with alpha level of 0.05 for main effects and 0.10 for marginal effects. Frequency and percent are presented for count data; mean, standard deviation, median, minimum and maximum are presented for continuous data. Logistic regression analysis with 60-day mortality as the outcome tested the effect of dose, with calculations performed using the natural logarithm of dose.

### Results

### A. Radiation Dose and Lethality

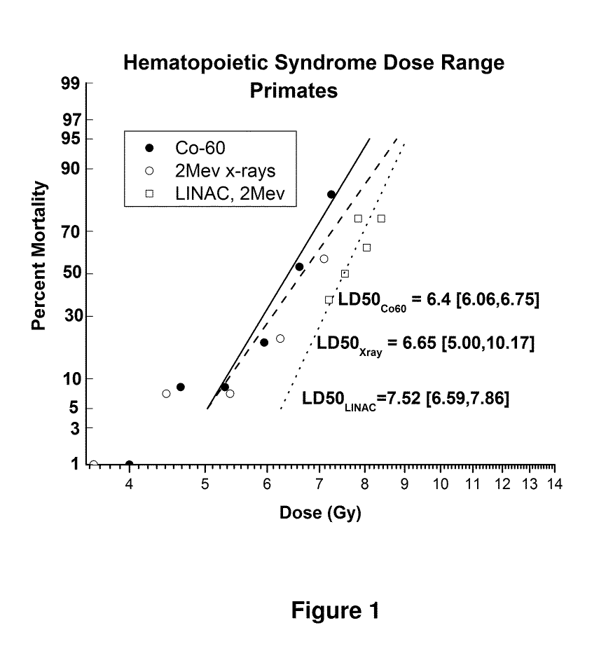

Forty-eight (48) male rhesus macaques were irradiated in seven cohorts (cohort 1, n=2; cohort 2, n=6; cohorts 3 thorough 7, n=8) over the dose range of 7.20 Gy to 8.90 Gy and administered medical management. Thirty-two (32) of 48 total animals (66.6%) succumbed to the hematopoietic syndrome. The dose response relationship is presented in FIG. 1 and in Table 1. Radiation dose was a significant predictor of mortality (P=0.01) with increased mortality rates at the higher doses.

TABLE 1
Percent Survival and Mean Survival Time
Following Radiation Exposure in Rhesus Macaques

| | | | | | | |
|---|---|---|---|---|---|---|
| Radiation Exposure | 7.20 | 7.55 | 7.85 | 8.05 | 8.40 | 8.90 |
| (Gy) | | | | | | |
| % Lethality | 38% | 50% | 75% | 63% | 75% | 100% |
| Decedents/total | 3/8 | 4/8 | 6/8 | 5/8 | 6/8 | 8/8 |

Survival time of decedents (days)

| | | | | | | |
|---|---|---|---|---|---|---|
| Mean | 20.0 | 18.3 | 22.2 | 16.2 | 17.5 | 21.1 |
| Median | 15.0 | 18.5 | 16.5 | 14.0 | 17.5 | 18.0 |

The estimated LD30/60, LD50/60, and LD70/60 values (with 95% CI in brackets) for rhesus monkeys exposed to TBI in this study were 7.09 Gy [6.50, 7.73], 7.52 Gy [7.12, 7.93], and 7.97 Gy [7.60, 8.36], respectively. Furthermore, estimation of the LD5/60 (6.24 Gy) [3.56, 6.91] and LD10/60 (6.51 Gy) [4.09, 7.09] relative to the LD95/60 (9.05 Gy) [8.45, 12.93] and LD90/60 (8.68 Gy) [8.22, 11.27] determined the respective ratios between the lethal doses for “few” and for “many” animals. The LD5:LD95 is 1.45 [1.24, 3.57] and LD10:LD90 is 1.33 [1.18, 2.70]. The respective difference in Gy between the “few” and “many” lethal events is approximately 2.81 to 2.17.

### B. Effect of Medical Management on the LD50.

As shown in FIG. 1, the LD50/30 from two historical studies available for rhesus macaques exposed to TBI of similar quality was estimated be 6.40 Gy (Co-60 γ-radiation, LD50_{Co60}) and 6.65 Gy (2 MV X-radiation, LD50_{Xray}) in the absence of supportive care (medical management). The value for the LD50/60 estimated from the current study employing TBI with 2 MV average LINAC photons plus medical management is 7.52 Gy. This retrospective comparison indicates that medical management will enhance the LD50 value and survival across the lethal hematopoietic syndrome radiation dosage range (FIG. 1 and Table 2).

The mean survival time (MST) of decedents at each radiation dose ranges from 16.2 days to 22.2 days (Table 1). The overall average MST across all doses for the study was 19.4 days Since a lethality dose-response study for animals not receiving medical management was not performed, the MST was calculated for all published dose response studies using rhesus macaques. This analysis yielded an average MST of 14.0 days across all known studies (Table 2).

TABLE 2
Total body irradiation and 60 day mortality:
Estimated LD30/60, LD50/60, and LD70/60 and MST of decedents
for all animals administered medical management
Lethal Doses for Hematopoietic Syndrome

| Pilot Study Estimate | |
|---|---|
| dose (Gy [95% CI]) | Literature Values* (Gy [95% CI]) |
| LD30/60 = 7.09 [6.50, 7.73] | LD50/30 = 6.40 [6.06, 6.75] Co-60 |
| LD50/60 = 7.52 [7.12, 7.93] | LD50/30 = 6.65 [5.00, 10.17] 2 |
| | MV x-rays |
| LD70/60 = 7.97 [7.60, 8.36] | |

Mean Survival Time (days)**
TBI, LINAC plus medical management = 19.4 days
TBI, Co-60, @ MV x-ray without medical management = 14.0 days
*One complete dose response study (2 MV x-ray) is available in the literature (Dalrymple, et al. 1965); the other (Co-60) was provided as a personal communication to Dr. MacVittie. No medical management was provided in these studies.
**The average MST of 14.0 days was calculated from all available literature determining the lethality dose response for rhesus macaques for hematopoietic syndrome without medical management.

Decedents that received medical management showed an average increase in MST of approximately 5.4 days compared to those that did not receive medical management. This observation is significant when considered in the context of administering a potential mitigator, such as a multi-PEGylated G-CSF variant of the invention (such as, for example, Maxy-G34) to lethally irradiated animals that are receiving effective medical management. In this case, the candidate mitigator would have the benefit of an additional 5 days to enhance marrow regeneration and production of mature cells such as neutrophils.

### C. Duration of Radiation-Induced Neutropenia.

Neutrophils provide the first line of defense against opportunistic infection. Lethal doses of TBI administered in this study reduced the circulating absolute neutrophil count (ANC) to 500/μL within approximately 5 days after TBI, irrespective of the radiation dose (Table 3).

TABLE 3
Duration of Cytopenia: Neutrophil-related parameters

| TBI | | | | | Recovery | | |
|---|---|---|---|---|---|---|---|
| Dose | First day ANC (d) | | Duration (d) | | to ANC | Days on | Nadir |
| (Gy) | <500/μL | <100/μL | <500/μL | <100/μL | ≧1000/μL | Antibiotics | (ANC/μL) |
| 7.20 | 4.6 ± 0.3 | 7.3 ± 0.3 | 11.5 | 11.5 | 23.74 | 19 | 0 |
| 7.55 | 5.5 ± 0.6 | 7.1 ± 0.4 | 24.0 | 9.8 | 26.7 | 28 | 0 |
| 7.85 | 4.6 ± 0.3 | 6.5 ± 0.4 | 14.3 | 10.3 | 21.7 | 18 | 5 |
| 8.05 | 5.0 ± 0.0 | 6.5 ± 0.3 | 15.0 | 10.3 | 22.3 | 11 | 0 |
| 8.40 | 5.0 | 6.4 | 19.0 | 15.0 | 42.0 | 19 | 0 |
| 8.90 | 4.6 | 6.0 | — | — | — | — | 0 |

* Duration (d) does not include data from decedent animals unless recovery occurred to that level, e.g., ANC ≧ 100/μL or ≧500/μL prior to death.

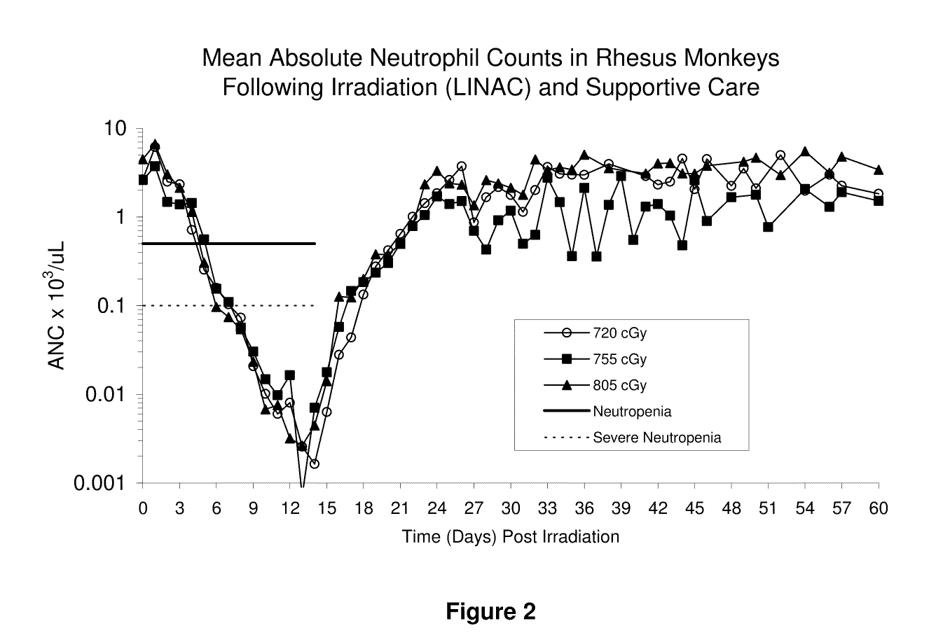

Antibiotics were administered when the ANC <500/μL because it was anticipated that the ANC might continue to decrease to values <100/μL. At severe Grade 4 neutropenia (ANC <100/μL) the animal is at greatest risk for infection and sepsis. Furthermore, these values determine the validity of administering primary antibiotic prophylaxis. The ANC in all lethally irradiated animals decreased to <100/μL within the next 1.5 to 3.0 days and continued to decrease in all dose cohorts with the exception of one (7.85 Gy), to absolute neutropenia (ANC ˜0/μL). The average nadir for the 7.85 Gy cohort was 5/μL (Table 3). The duration of Grade 4 neutropenia (ANC <100/μL) for survivors, over all dose cohorts, ranged from 9.8 to 15.0 days where the range over all dose cohorts for the duration of ANC <500/μL was 11.5 to 24.0 days. Additional neutrophil-related parameters are shown in Table 3. Shown in FIG. 2 are the neutrophil recovery curves for all animals exposed to doses of TBI that approximate the LD30/60, LD50/60, and LD70/60 levels.

In conclusion, this study demonstrates that the dose of uniform TBI with average 2 MV LINAC photons was a significant predictor of lethality. The doses of TBI used herein permitted estimation of LD30/60, LD50/60, and LD70/60 levels for the design of efficacy trials for agents that mitigate the lethality associated with the hematopoietic syndrome of ARS. In this study, the LD30/60, LD50/60, and LD70/60 levels were 7.09, 7.52 and 7.97 Gy, respectively. Compared to literature values determined in studies designed to assess the lethal radiation dose response of rhesus macaques without the benefit of medical management, medical management (as administered in the study presented herein) increased the LD50/60 associated with the hematopoietic syndrome of ARS, and increased the MST of decedents.

### Example 2

### Pharmacodynamics and Pharmacokinetics of a Multi-PEGylated G-CSF Variant in a Non-Human Primate Model of Radiation-Induced Neutropenia

### Study Protocol:

The studies were conducted according to the principles enunciated in the Guide for the Care and Use of Laboratory Animals (The Institute of Laboratory Animal Resources, National Research Council, 1996). Male rhesus monkeys (Macaca mulatta) with a mean weight of 4.6+/−0.7 kg were exposed to 250 kVp X-irradiation at 0.13 Gy/min unilaterally in the posterior-anterior position, and rotated 108E at mid-dose (3.00 Gy) to the anterior-posterior position for the completion of the total 6.00 Gy exposure. Animals received clinical support, consisting of antibiotics, fresh irradiated whole blood, and fluids, as needed. Gentamicin (Elkin Sinn, Cherry Hill N.J.) was administered intramuscularly (i.m.) every day (q.d.) at 10 mg/day for the first seven days of treatment. Baytril® (Bayer Corp., Shawnee Mission, Kans.) was administered 10 mg/day i.m. q.d. for the entire period of antimicrobial treatment. Antibiotics were administered until the animal maintained a WBC ≧1,000/μl for 3 consecutive days and had attained and ANC ≧500/μl. Animals received fresh, irradiated (15.00 Gy Co⁶⁰-irradiated) whole blood, approximately 30 ml/transfusion, from a random donor pool of monkeys when the platelet (PLT) count was <20,000/μl and the hematocrit (HCT) was <18%.

Nine irradiated and two non-irradiated male Rhesus moneys were treated with an exemplary multi-PEGylated G-CSF variant according to the invention (identified herein as “Maxy-G21”), and four irradiated Rhesus macaques were treated with Neulasta®. Four animals treated with diluent only (“vehicle”) served as controls. In the Neulasta® group two animals were sampled for PK analysis, whereas all the Maxy-G21-treated animals were included in the pharmacokinetic assessment. Each animal was administered a single subcutaneous dose of the test compound or vehicle 24 hours after total body irradiation. Two different dosages of Maxy-G21 were employed: 100 and 300 μg per kg, employing 4 and 5 monkeys, respectively. The Neulasta® group was administered 300 μg/kg. Two non-irradiated animals administered 300 μg/kg Maxy-G21 were used for studying mobilization of CD34+ cells and in vitro colony forming cells (CFC). Blood samples were collected from the saphenous vein. An overview of the study design is provided in Table 4.

TABLE 4
Summary of Study Protocol

| Drug | Dose (μg/kg) | Number of animals | Route |
|---|---|---|---|
| Vehicle | N/A | 4 | s.c. |
| Maxy-G21 | 300 | 5 | s.c. |
| Maxy-G21 | 300 | 4 | s.c. |
| Maxy-G21* | 100 | 2 | s.c. |
| Neulasta | 300 | 4 | s.c. |

*These animals were used for studying mobilization of CD34+ cells and in vitro colony forming cells (CFC)

### Results:

Compared to control animals exposed to 6.00 Gy TBI dosed with autologous serum (AS), which exhibited a period of neutropenia of 14.8-15.7 days, irradiated animals dosed with 300 μg/kg Maxy-G21 exhibited a shortened the period of neutropenia of 7.3±1.1 days. The ANC nadir was also markedly improved to 140±45/μL, from as low as 49±22/μL, in control animals. Time to recovery was likewise improved from a control value of 21.2±0.4 and 23.0±0.0 days (in three separate control cohorts), to 15.5±0.3 days in the Maxy-G21 treated cohort. As compared to the intra-study Neulasta® cohort (N=4) employing an equivalent dose of Neulasta® (300 μg/kg), Maxy-G21 was found to reduce the duration of neutropenia by 2 days (from 9.3 to 7.3 days), the time to recovery by 3 days (from 18.5 to 15.5 days) and the antibiotic requirement by 3 days (from 11.5 to 9.8 days; Table 5).

TABLE 5
The effect of Maxy-G21 administration on neutrophil-related parameters
in 6.00 Gy x-irradiated rhesus macaques versus treatment with Neulasta ®
or control autologous sera (AS):
Neutropenic duration, nadir, time to recovery and clinical support

| | | | | Time to | Antibiotic |
|---|---|---|---|---|---|
| | | Duration of | ANC nadir | recovery | requirements |
| Treatment groups | n | neutropenia (days) | (per μL) | (days) | (days) |
| Control cohorts: | | | | | |
| AS FY01-02* | 11 | 14.8 ± 0.6 | 49 ± 22 | 21.2 ± 0.4 | 16.8 ± 0.6 |
| AS FY02-03* | 7 | 15.2 ± 0.6 | 80 ± 30 | 22.0 ± 0.3 | 15.4 ± 0.3 |
| AS This study | 4 | 15.7 ± 0.8 | 109 ± 37 | 23.0 ± 0.0 | 16.0 ± 0.0 |
| Maxy-G21 | 4 | 7.3 ± 1.1 | 140 ± 45 | 15.5 ± 0.3 | 9.8 ± 1.5 |
| Neulasta ® cohorts: | | | | | |
| This study | 4 | 9.3 ± 1.5 | 135 ± 16 | 18.5 ± 1.7 | 11.5 ± 2.4 |
| Historical | 5 | 14.4 ± 1.4 | 80 ± 24 | 21.2 ± 1.6 | 17.2 ± 1.2 |
| **Neulasta ® | 9 | 12.1 ± 1.3 | 104 ± 17 | 20.0 ± 1.2 | 14.7 ± 1.5 |

*Separate control cohorts
**Combined Neulasta ® cohort comprising this study and a published cohort from the MacVittie laboratory.

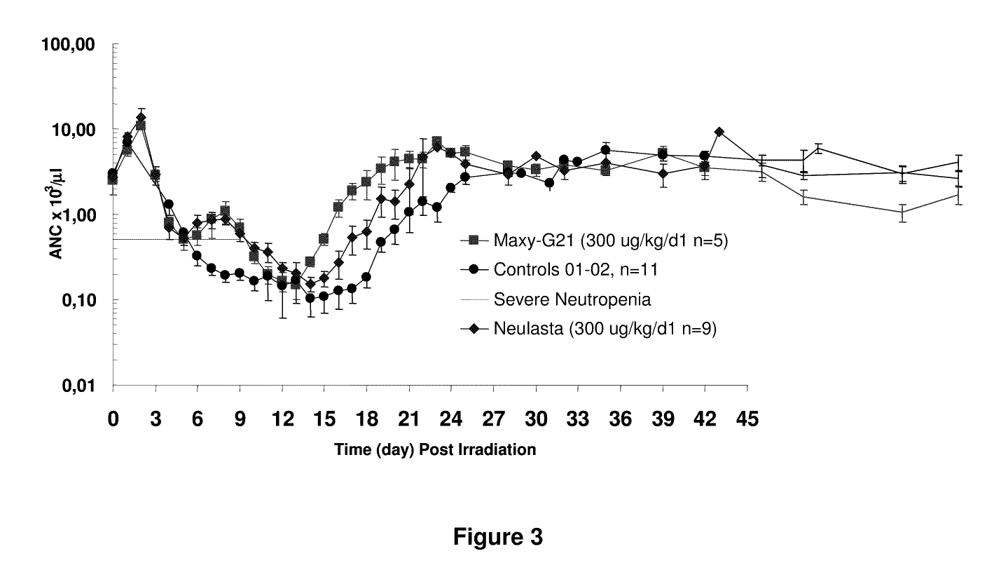

When the data from a historical Neulasta® cohort (n=5) was combined with the current intra-study Neulasta® cohort (n=4), the duration of neutropenia was 12.1±1.3 days. The duration of neutropenia was significantly shorter in the Maxy-G21 group in comparison to the combined Neulasta® group (P=0.02) (FIG. 3, Table 5). The control and Maxy-G21-treated cohort only required antibiotics for 9.8 days, whereas the combined Neulasta®-treated cohort required 14.7 days of antibiotic support. Maxy-G21 administered at 100 μg/kg (data not shown) was not effective in stimulating neutrophil recovery under the conditions of this study, as assessed by all neutrophil-related parameters.

After subcutaneous administration of Maxy-G21, the drug reached peak plasma concentration within 24 to 96 hours in both irradiated and non-irradiated Rhesus macaques. In the two 300 μg/kg dosage groups the peak plasma concentrations were roughly three times higher than in the 100 μg/kg group. A biphasic Maxy-G21 elimination pattern is seen in both the normal and irradiated animals treated with 300 μg/kg of drug (FIG. 4).

The irradiated animals treated with 300 μg/kg Maxy-G21 exhibited an early-slow elimination phase with a mean serum half-life of 59 hours. The duration of the early-slow phase was 12-13 days (FIG. 4). The early profiles are characterized by uniformity among the 5 macaques. At day 15 after injection of the drug substance the slow phase is superseded by a faster phase, which showed a mean plasma half-life of 16 hours. The late elimination phase, based on the analysis of data from 3 animals, was characterized by more inter-animal variation in plasma half-lives. In the irradiated animals treated with 100 μg/kg Maxy-G21, the drug was eliminated in a single phase with a mean serum half-life of 49 hours.

Non-irradiated (“normal”) animals eliminated Maxy-G21 (300 μg/kg) in a fast-early and slower-late phase kinetic profile. The mean plasma half-life of the late phase was 62 hours as compared to less than 35 hours for Neulasta® in non-irradiated animals in a published study (data not shown). A comparison of non-irradiated and irradiated animals shows a 3-fold difference in AUC at the same dose of 300 μg/kg of Maxy-G21 (Table 6).

Neulasta® was found to be eliminated in a single phase with a mean plasma half-life of 23 hours, which is markedly faster than observed for Maxy-G21 (FIG. 4). The peak plasma concentration of Neulasta® was found to be 5-6 times lower as compared to Maxy-G21 (Table 6). After 11 to 15 days, Neulasta® was undetectable in plasma. AUC for Neulasta® was approximately 9-10 times lower as compared to Maxy-G21.

TABLE 6
Pharmacokinetics of Maxy-G21 and Neulasta ®-treated
irradiated rhesus monkeys rhesus monkeys
and Maxy-G21 treated non-irradiated rhesus monkeys.
Values represent mean ± sd.

| | | | Maxy-G21 | |
|---|---|---|---|---|
| | Maxy-G21 | Maxy-G21 | 300 μg/kg | Neulasta ® |
| | 300 μg/kg | 100 μg/kg | Non- | 300 μg/kg |
| PK parameters | Irradiated | Irradiated | Irradiated | Irradiated |
| Cₘₐₓ (ng/mL) | 7219 ± 1476 | 1961 ± 172 | 5953 ± 490 | 1239 ± 658 |
| Tₘₐₓ (hrs) | 53 ± 31 | 50 ± 4 | 31 ± 0 | 15 ± 13 |
| AUC (hrs/mL) | 928609 ± 88114 | 165798 ± 45705 | 359040 ± 18837 | 198684 ± 124275 |

### Conclusions:

The present study provides evidence that an exemplary multi-PEGylated G-CSF variant according to the invention (identified herein as Maxy-G21) administered s.c. to rhesus macaques is capable of significantly shortening the period of neutropenia in radiation-induced neutropenic NHP. The effect was furthermore found to exceed that of Neulasta® when compared to a cohort comprising the intra-study Neulasta® cohort and a historical Neulasta® cohort (N=9).

Overall, the exemplary multi-PEGylated G-CSF variant Maxy-G21 exhibited a markedly extended plasma half-life as compared to the mono-PEGylated hG-CSF Neulasta® in NHP undergoing a state of severe radiation-induced myelosuppression as well as in healthy (non-irradiated) non-human primates. The PK data supports the working hypothesis that multi-PEGylated G-CSF variants such as Maxy-G21 have greater bioavailability and a sustained duration of action relative to mono-PEGylated Neulasta® during a state of severe radiation-induced myelosuppression, as well as in normal (non-irradiated) NHP.

### Example 3

### Radiomitigating Activity of a Subcutaneously Administered Multi-PEGylated G-CSF Variant after Lethal Radiation Exposure in C57BL/6 Mice

The efficacy of an exemplary multi-PEGylated G-CSF variant (identified herein as Maxy-G34) was tested at a 1 mg/kg dosage and at two different lethal doses of radiation. Mice at each radiation dose level were apportioned into treatment groups of 20 mice each (10 females and 10 males) receiving Maxy-G34 on days 1, 7, and 14 or days 1 and 7 following irradiation at 7.76 Gy or at 7.96 Gy. Vehicle-treated mice received diluent (a sterile liquid solution of 10 mM sodium acetate, 45 mg/ml mannitol, 0.05 mg/ml polysorbate 20, pH 4.0) on days 1, 7, and 14. Thus, the three groups of mice received one of the following treatments:
1. Maxy-G34; 24±4 hr and 7 d±4 hr after 7.76 Gy irradiation (Maxy-G34 d1, d7)
2. Maxy-G34; 24±4 hr, 7 d±4 hr and 14 d±4 hr after 7.76 Gy irradiation (Maxy-G34 d1, d7, d14)
3. Vehicle; 24±4 hr, 7 d±4 hr and 14 d±4 hr after 7.76 Gy irradiation (Vehicle d1, d7, d14)
4. Maxy-G34; 24±4 hr and 7 d±4 hr after 7.96 Gy irradiation (Maxy-G34 d1, d7)
5. Maxy-G34; 24±4 hr, 7 d±4 hr and 14 d±4 hr after 7.96 Gy irradiation (Maxy-G34 d1, d7, d14)
6. Vehicle; 24±4 hr, 7 d±4 hr and 14 d±4 hr after 7.96 Gy irradiation (Vehicle d1, d7, d14)
   The mice were irradiated in groups of 14-16 animals, at the following doses:

7.76 Gy: 66.104 cGy/min (11 min 44 sec exposure time)

7.96 Gy: 66.104 cGy/min (12 min 02 sec exposure time)

The mice were not administered antibiotics. The primary endpoint was 30 day overall survival, and the secondary endpoint was mean survival time (MST).

### Results:

Survival over 30 days and mean survival times (MST) are shown in Table 7, Table 8, FIG. 5 and FIG. 6.

TABLE 7
Thirty-Day Survival and MST

| | | | | | Mean |
|---|---|---|---|---|---|
| | Rad | | No. of | | Survival |
| | dose | | Survivors/ | Percent | Time of |
| Group | (Gy) | Group Description | Total | Survival | Descendents |
| 1 | 7.76 | Maxy-G34 d1, d7 | 19/20 | 95 | 16.0 |
| 2 | 7.76 | Maxy-G34 d1, d7, | 19/20 | 95 | 12.0 |
| | | d14 | | | |
| 3 | 7.76 | Vehicle d1, d7, d14 | 16/20 | 80 | 17.5 |
| 4 | 7.96 | Maxy-G34 d1, d7 | 15/20 | 75 | 12.6 |
| 5 | 7.96 | Maxy-G34 d1, d7, | 17/20 | 85 | 8.3 |
| | | d14 | | | |
| 6 | 7.96 | Vehicle d1, d7, d14 | 11/20 | 55 | 15.1 |

TABLE 8
Statistical analysis of Survival and Mean Survival Time
(pooled data of 7.76Gy and 7.96Gy)

| | One-sided |
|---|---|
| Comparison | p-Value |
| 30 day survival of “Maxy-G34 d1, d7” | 0.0499 |
| compared to “Vehicle d1, d7, d14” | |
| 30 day survival of “Maxy-G34 d1, d7, d14” | 0.017 |
| compared to “Vehicle d1, d7, d14” | |
| MST of “Maxy-G34 d1, d7” compared to | Not significant |
| “Vehicle: d1, d7” | |
| MST of “Maxy-G34: d1, d7, d14” compared | Not significant |
| to “Vehicle: d1, d7, d14” | |

Irradiation of mice at 7.76 Gy radiation dose followed by treatment with vehicle at d1, d7 and d14 post exposure resulted in 80% survival after 30 days (i.e., LD20/30). Treatment of the 7.76 Gy (LD20/30) irradiated mice with 1 mg/kg Maxy-G34 at d1, d7 and d14 post exposure, or at d1 and d7 post exposure, both increased survival after 30 days to 95%. (Table 7).

At the 7.96 Gy radiation dose, survival in the vehicle d1, d7, d14 group was 55% 30 days post-exposure (i.e., LD 45/30). The 7.96 Gy (LD45/30) irradiated mice treated with 1 mg/kg Maxy-G34 at d1 and d7 showed 75% survival 30 days post-exposure, and the Maxy-G34 d1, d7, d14 group showed 85% survival 30 days post-exposure. At this radiation dose level, the 3-week dose regimen (d1, d7, d14) appeared to be more effective than the 2-week dose regimen (d1, d7).

The data obtained from the two levels of radiation were combined (Table 8). Under the conditions of this study, both the 3-week Maxy-G34 dose group and the 2-week Maxy-G34 dose group showed statistically significant increases in survival 30 days post irradiation over that of the vehicle control groups. At the radiation dosages employed in this study, the differences in MST between the treatment groups and the vehicle control groups were not statistically significant.

While the foregoing invention has been described in some detail for purposes of clarity and understanding, it will be clear to one skilled in the art from a reading of this disclosure that various changes in form and detail can be made without departing from the true scope of the invention. It is understood that the examples and embodiments described herein are for illustrative purposes only and that various modifications or changes in light thereof will be suggested to persons skilled in the art and are to be included within the spirit and purview of this application and scope of the appended claims. All publications, patents, patent applications, and/or other documents cited in this application are incorporated herein by reference in their entirety for all purposes to the same extent as if each individual publication, patent, patent application, and/or other document were individually indicated to be incorporated herein by reference in its entirety for all purposes.

We claim:

1. A method for treating or preventing neutropenia in a patient subjected to radiation exposure, comprising administering to the patient after the radiation exposure a multi-PEGylated G-CSF variant, wherein the multi-PEGylated G-CSF variant comprises:
a polypeptide exhibiting G-CSF activity, the polypeptide comprising an amino acid sequence that differs in up to 15 amino acid residues from the amino acid sequence shown in SEQ ID NO:1, and
two or more polyethylene glycol (PEG) moieties, each PEG moiety covalently attached either directly or indirectly to an amino acid residue of the polypeptide.

2. The method of claim 1, wherein the multi-PEGylated G-CSF variant comprises the amino acid sequence of SEQ ID NO:1 and at least one substitution relative to SEQ ID NO: 1 selected from the group consisting of T1K, P2K, L3K, G4K, P5K, A6K, S7K, S8K, L9K, P10K, Q11K, S12K, F13K, L14K, L15K, E19K, Q20K, V21K, Q25K, G26K, D27K, A29K, A30K, E33K, A37K, T38K, Y39K, L41K, H43K, P44K, E45K, E46K, V48K, L49K, L50K, H52K, S53K, L54K, 156K, P57K, P60K, L61K, S62K, S63K, P65K, S66K, Q67K, A68K, L69K, Q70K, L71K, A72K, G73K, S76K, Q77K, L78K, S80K, F83K, Q86K, G87K, Q90K, E93K, G94K, S96K, P97K, E98K, L99K, G100K, P101K, T102K, D104K, T105K, Q107K, L108K, D109K, A111K, D112K, F113K, T115K, T116K, W118K, Q119K, Q120K, M121K, E122K, E123K, L124K, M126K, A127K, P128K, A129K, L130K, Q131K, P132K, T133K, Q134K, G135K, A136K, M137K, P138K, A139K, A141K, S142K, A143K, F144K, Q145K, S155K, H156K, Q158K, S159K, L161K, E162K, V163K, S164K, Y165K, V167K, L168K, H170K, L171K, A172K, Q173K and P174K.

3. The method of claim 2, wherein the amino acid sequence of the multi-PEGylated G-CSF variant comprises at least one substitution selected from the group consisting of Q70K, Q90K, T105K Q120K, T133K, S159K and H170K.

4. The method of claim 2, wherein the amino acid sequence of the multi-PEGylated G-CSF variant further comprises at least one substitution selected from the group consisting of K16R/Q, K34R/Q, and K40R/Q.

5. The method of claim 3, wherein the amino acid sequence of the multi-PEGylated G-CSF variant comprises the substitutions K16R, K34R, K40R, T105K and S159K.

6. The method of claim 5, wherein the amino acid sequence of the multi-PEGylated G-CSF variant consists of the substitutions K16R, K34R, K40R, T105K and S159K and optionally a methionine reside at the N-terminus.

7. The method of claim 1, wherein the multi-PEGylated G-CSF variant comprises 2-6 PEG moieties each with a molecular weight of about 1-10 kDa.

8. The method of claim 7, wherein the multi-PEGylated G-CSF variant comprises a PEG moiety attached to the N-terminus and a PEG moiety attached to a lysine residue.

9. The method of claim 7, wherein the multi-PEGylated G-CSF comprises 2-4 PEG moieties each with a molecular weight of about 4-6 kDa.

10. The method of claim 1, wherein the amino acid sequence of the multi-PEGylated G-CSF variant comprises one or more substitution selected from K16R/Q, K34R/Q, and K40R/Q and one or more substitution selected from Q70K, Q90K, T105K, Q120K, T133K, and S159K, and comprises 2-6 attached PEG moieties each with a molecular weight of about 1-10 kDa.

11. The method of claim 10, wherein the amino acid sequence of the multi-PEGylated G-CSF variant comprises one or more substitution selected from K16R/Q, K34R/Q, and K40R/Q and at least one substitution selected from T105K and S159K, and comprises 2-4 attached PEG moieties each with a molecular weight of about 1-10 kDa.

12. The method of claim 11, wherein the amino acid sequence of the multi-PEGylated G-CSF variant comprises the substitutions K16R, K34R, K40R, T105K and S159K, and comprises 2-4 attached PEG moieties each with a molecular weight of about 4-6 kDa.

13. The method of claim 13, wherein the multi-PEGylated G-CSF variant is a mixture of positional PEG isomer species.

14. The method of claim 13, wherein the mixture of positional PEG isomer species comprises at least 2 species of positional PEG isomers each having 3 attached PEG moieties, wherein one of the isomers has PEG moieties attached at the N-terminal, Lys23 and Lys 159, and the other isomer has PEG moieties attached at the N-terminal, Lys 105 and Lys 159.

15. The method of claim 14, wherein the PEG moieties each have a molecular weight of about 1-10 kDa.

16. The method of claim 15, wherein the PEG moieties each have a molecular weight of about 5 kDa.

17. The method of claim 1, wherein the multi-PEGylated G-CSF variant exhibits an improved pharmacokinetic property compared to Neulasta® (pegfilgrastim) when tested under comparable conditions in an animal model.

18. The method of claim 17, wherein the multi-PEGylated G-CSF variant exhibits an increased serum half-life compared to Neulasta® in an animal model.

19. The method of claim 17, wherein the multi-PEGylated G-CSF variant exhibits an increased AUC compared to Neulasta® in an animal model.

20. The method of claim 1, wherein the multi-PEGylated G-CSF variant is administered to the patient in an amount effective to reduce the duration of severe neutropenia in a group treated with the multi-PEGylated G-CSF variant relative to a group not treated with the multi-PEGylated G-CSF variant in an animal model system of radiation-induced neutropenia.

21. The method of claim 1, wherein the multi-PEGylated G-CSF variant is administered to the patient in an amount effective to increase the number of survivors 30 days post-radiation exposure in a group treated with the multi-PEGylated G-CSF variant relative to a group not treated with the multi-PEGylated G-CSF variant in an animal model system of radiation-induced neutropenia.

22. The method of claim 1, wherein the multi-PEGylated G-CSF variant is administered to the patient in a dose of from about 20 ug/kg patient weight to about 300 ug/kg patient weight.

23. The method of claim 1, wherein the patient is an adult human and the multi-PEGylated G-CSF variant is administered to the patient in a dose of from about 1-30 mg per patient.

24. The method of claim 1, wherein one or more additional hematopoietic growth factor is administered.

25. The method of claim 24, wherein the additional hematopoietic growth factor is selected from granulocyte macrophage colony stimulating factor (GM-CSF), stem cell factor (SCF), FLT3-ligand (FL), interleukin-3 (IL-3), megakaryocyte growth and development factor (MGDF), thrombopoietin (TPO), a TPO-receptor agonist, and erythropoietin (EPO).

26. The method of claim 1, wherein the multi-PEGylated G-CSF variant is administered to the subject within about 3 days after the radiation exposure.

27. The method of claim 1, wherein the radiation exposure is equal to or greater than about 1 Gy.


SEQUENCE LISTING (1 sequence)

SEQ ID NO: 1 - protein, 174 aa, Homo sapiens
TPLGPASSLPQSFLLKCLEQVRKIQGDGAALQEKLCATYKLCHPEELVLLGHSLGIPWAPLSSCPSQALQLAGCLSQLHSGLFLYQGLLQALEGISPELGPTLDTLQLDVADFATTIWQQMEELGMAPALQPTQGAMPAFASAFQRRAGGVLVASHLQSFLEVSYRVLRHLAQP